(19) United States
(12) Patent Application Publication
(10) Pub. No.: US 2006/0205081 A1
(43) Pub. Date: Sep. 14, 2006

(54) Use of methyltransferase inhibitors to enhance transgene expression

(76) Inventors: Linhong Li, North Potomac, MD (US); Cornell Allen, Woodlawn, MD (US)

Correspondence Address: FULBRIGHT & JAWORSKI L.L.P., 600 CONGRESS AVE., SUITE 2400, AUSTIN, TX, 78701, US

(21) Appl. No.: 11/357,365

(22) Filed: Feb. 17, 2006

Related U.S. Application Data
(60) Provisional application No. 60/654,216, filed on Feb. 18, 2005.

(51) Int. Cl. C12N 15/87

(52) U.S. Cl. 435/461

(57) ABSTRACT

The present invention concerns enhancing transgene expression in electroporated cells. Electroporation-mediated transfection of cells has several advantages over other transfection methods. However, the efficiency of transgene expression in electroporated cells may be lower than desired for certain applications. The ability to further enhance transgene expression in electroporated cells would be useful in a number of applications including protein and virus production. The present invention provides a method for enhancing transgene expression in a cell, the method comprising transfecting the cell by electroporation with an expression construct encoding a protein; and contacting the cell with a methyltransferase inhibitor, wherein the expression of the protein is enhanced as compared to the expression of the protein in a second cell not contacted with the methyltransferase inhibitor.

This application claims priority to U.S. Provisional Application No. 60/654,216, filed Feb. 18, 2005, which is incorporated herein in its entirety.

### BACKGROUND OF THE INVENTION

1. Field of the Invention

The present invention relates generally to the field of molecular biology. More particularly, it concerns transgene expression in transfected cells.

2. Description of Related Art

Electroporation-mediated transfection of cells has several advantages over other transfection methods. For example, electroporation avoids the risks associated with virus-mediated transfection, and it can reduce labor and costs. However, the efficiency of transgene expression in electroporated cells may be lower than desired for certain applications. The efficiency of transgene expression in transfected cells can be limited by the duration of transgene expression and/or the level of transgene expression.

The mechanism by which transgene expression is limited is not completely understood. A potential factor is the methylation of the expression construct. DNA methylation is known to be involved in regulating endogenous gene expression in cells. The degree of DNA methylation reflects the state of a gene's transcriptional activity, with hypomethylation being correlated with increased transcription and hypermethylation being correlated with decreased expression. Inhibiting DNA methylation has been shown to increase the expression of a reporter gene (LacZ) in biolistically transfected mouse cells (D'Angelo et al., 1999) and calcium phosphate transfected CHO cells (MacGregor et al., 1987). The ability to further enhance transgene expression in electroporated cells would be useful in a number of applications including protein and virus production.

### SUMMARY OF THE INVENTION

In one embodiment, the present invention provides a method for enhancing transgene expression in a eukaryotic cell, the method comprising: transfecting a eukaryotic cell by electroporation with an expression construct encoding a transgene; and contacting the cell with a demethylating agent, wherein the expression of the transgene is enhanced as compared to the expression of the transgene in a second eukaryotic cell not contacted with a demethylating agent.

In another embodiment, the invention provides a method for enhancing transgene expression in a eukaryotic cell, the method comprising: transfecting a eukaryotic cell by electroporation with an expression construct encoding a transgene; and contacting the cell with a methyltransferase inhibitor, wherein the expression of the transgene is enhanced as compared to the expression of the transgene in a second eukaryotic cell not contacted with a methyltransferase inhibitor. As used herein, “enhancing transgene expression” means increasing the level of transgene expression and/or prolonging transgene expression.

In some embodiments, the transgene may encode a peptide, polypeptide, or protein. In other embodiments, the transgene encodes a non-protein coding RNA, such as a ribosomal RNA, tRNA, splicosomal RNA, antisense RNA, siRNA, or mRNA.

The present invention may be used to enhance transgene expression in any eukaryotic cell in which DNA is methylated. In some embodiments the eukaryotic cell is a mammalian cell. Examples of preferred mammalian cells include human, mouse, hamster, and rat cells. In other embodiments the eukaryotic cell is a plant cell. The transgene may be integrated into the genomic DNA of the host cell or it may be extrachromosomal.

In one embodiment, the invention provides a method for enhancing transgene expression in a eukaryotic cell, the method comprising: transfecting a eukaryotic cell by electroporation with an expression construct encoding a polypeptide; and contacting the cell with a methyltransferase inhibitor, wherein the expression of the polypeptide is enhanced as compared to the expression of the polypeptide in a second eukaryotic cell not contacted with a methyltransferase inhibitor. As used herein, “enhancing transgene expression” means increasing the level of transgene expression and/or prolonging transgene expression.

Any method of transfecting cells by electroporation known in the art may be used in the present invention. In certain aspects of the invention the electroporation is static electroporation. In other aspects of the invention, the electroporation is flow electroporation.

The cell may be contacted with the methyltransferase inhibitor before, during, or after transfection, or a combination thereof. In one embodiment, the cell is contacted with the methyltransferase inhibitor during transfection with the expression construct. In a preferred embodiment, the cell is contacted with the methyltransferase inhibitor following transfection with the expression construct. In another preferred embodiment, the cell is contacted with the methyltransferase inhibitor before transfection with the expression construct. In other preferred embodiments, the cell is contacted with the methyltransferase inhibitor both before and after transfection with the expression construct.

Typically, the methyltransferase inhibitor will be added to the culture medium. The concentration of methyltransferase inhibitor will vary depending on the cell type, the particular methyltransferase inhibitor, and the level of expression desired. For example the concentration may be about 0.005, 0.008, 0.01, 0.04, 0.08, 0.1, 0.2, 0.25, 0.4, 0.5, 0.75, 1.0, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 12, 14, 16, 18, 20, or 25 μM, or any range derivable therein. It is also envisioned that the methyltransferase inhibitor may be incorporated into the vector prior to transfection into the cell.

The amount of time the cell is contacted with a methyltransferase inhibitor can also vary depending on the effect desired. In certain aspects of the invention the cell is contacted with the methyltransferase inhibitor for about 4, 6, 12, 18, 24, 30, 36, 42, 48, 54, 60, 66, 72, 80, 86, 92, 98, 104, 110, 116, or 124 hours, or any range derivable therein. In other aspects of the invention, the transfected cell is maintained indefinitely in culture medium comprising the methyltransferase inhibitor.

Numerous inhibitors of DNA methylation are known in the art. Examples of methyltransferase inhibitors include 5-azacytidine, 5-aza-2′-deoxycytidine, L-ethionine, dihydro-5-azacytidine, arabinofuranosyl-5-azacytosine (fazarabine), and 2-pyrimidone-1-β-D-riboside-1-(β-D-ribofuranosyl)-1,2-dihydropyrimidin-2-one (Zebularine). Inhibitory nucleic acid sequences, such as siRNA or mRNA, directed to DNA methyltransferase genes could also be used to inhibit DNA methylation. Inhibitory nucleic acid sequences, targeting other proteins involved in silencing methylated DNA could also be used. Examples of DNA methyltransferase genes include Dnmt-1, Dnmt-2, Dnmt-3, and Dnmt3B. In certain embodiments of the invention an siRNA, mRNA, antisense or other inhibitory nucleic acid or a nucleic acid, which may be either RNA or DNA, that codes for such an inhibitor nucleic acid targeting a DNA methyltransferase gene is also loaded into the cell with a transgene of interest in order to enhance transcription of the transgene. In other embodiments, an expression construct encoding an siRNA, mRNA, or other inhibitory nucleic acid targeting a DNA methyltransferase gene is co-transfected with a transgene of interest in order to enhance transcription of the transgene. In other embodiments, a an antibody or antibody fragment or antibody-like molecule which inhibits the activity of a DNA methyltransferase or a nucleic acid, which may be either RNA or DNA, that codes for an antibody or antibody fragment or antibody-like molecule which inhibits a DNA methyltranseferase is also loaded into the cell with a transgene of interest to enhance transcription of the transgene. It is contemplated that any methyltransferase inhibitor may be used with the present invention. It is also contemplated that 2, 3, 4, 5, or more methyltransferase inhibitors may be used in combination. It is further contemplated that 1, 2, 3, 4, or more expression constructs encoding 1, 2, 3, 4, or more transgenes of interest may be delivered by electroporation essentially simultaneously to a host cell.

In some embodiments, the expression construct encodes a cytosolic protein, a membrane protein, or a secreted protein. The protein may be a therapeutic protein. A “therapeutic protein” is a protein that can be administered to a subject for the purpose of treating or preventing a disease. Examples of classes of therapeutic proteins include tumor suppressors, inducers of apoptosis, cell cycle regulators, immuno-stimulatory proteins, toxins, cytokines, enzymes, antibodies, inhibitors of angiogenesis, angiogenic factors, growth factors, metalloproteinase inhibitors, hormones or peptide hormones. The therapeutic protein may be isolated from the cell from which it was produced prior to administering it to a subject. Alternatively, the transfected cell expressing the therapeutic protein may be administered to a subject.

An “immuno-stimulatory protein” is a protein involved in the activation, chemotaxis, or differentiation of immune cells. Examples of classes of immuno-stimulatory proteins include thymic hormones, cytokines, and growth factors. Thymic hormones include, for example, prothymosin-α, thymulin, thymic humoral factor (THF), THF-γ-2, thymocyte growth peptide (TGP), thymopoietin (TPO), thymopentin, and thymosin-α-1. Examples of cytokines include, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, leukocyte inhibitory factor (LIF), IFN-α, IFN-β, IFN-γ, TNF, TNF-α, TGF-β, G-CSF, M-CSF, and GM-CSF. Other immuno-stimulatory proteins include B7.1 (CD80), B7.2 (CD86), ICAM-1 (CD54), VCAM-1, LFA-1, VLA-4, CD40, and CD40L (CD154).

Examples of other proteins contemplated by the present invention include developmental proteins such as adhesion molecules, cyclin kinase inhibitors, Wnt family members, Pax family members, Winged helix family members, Hox family members, cytokines/lymphokines and their receptors, growth or differentiation factors and their receptors, neurotransmitters and their receptors; oncogenes such as ABL1, BLC1, BCL6, CBFA1, CBL, CSFIR, ERBA, ERBB, EBRB2, ETS1, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, and YES; tumor suppressors such as p53, Rb, Rap1A, DCC, k-rev, BRCA1, BRCA2, zac1, p73, MMAC-1, ATM, HIC-1, DPC-4, FHIT, APC, DCC, PTEN, ING1, NOEY1, NOEY2, PML, OVCA1, MADR2, WT1, 53BP2, IRF-1, MADH4, MCC, NF1, NF2, RB1, TP53, and WT1; enzymes such as carbamoyl synthetase I, ornithine transcarbamylase, arginosuccinate synthetase, arginosuccinate lyase, arginase, ACP desaturases and hycroxylases, ADP-glucose pyrophorylases, ATPases, alcohol dehydrogenases, amylases, amyloglucosidases, catalases, cellulases, cyclooxygenases, decarboxylases, dextrinases, esterases, DNA and RNA polymerases, hyaluron synthases, galactosidases, glucanases, glucose oxidases, GTPases, helicases, hemicellulases, hyaluronidases, integrases, invertases, isomersases, kinases, lactases, lipases, lipoxygenases, lyases, lysozymes, pectinesterases, peroxidases, phosphatases, phospholipases, phophorylases, polygalacturonases, proteinases and peptideases, pullanases, recombinases, reverse transcriptases, topoisomerases, and xylanases; and hormones such as growth hormone, prolactin, placental lactogen, luteinizing hormone, follicle-stimulating hormone, chorionic gonadotropin, thyroid-stimulating hormone, leptin, adrenocorticotropin (ACTH), angiotensin I and II, β-endorphin, β-melanocyte stimulating hormone (β-MSH), cholecystokinin, endothelin I, galanin, gastric inhibitory peptide (GIP), glucagon, insulin, lipotropins, neurophysins, somatostatin, calcitonin, calcitonin gene related peptide (CGRP), β-calcitonin gene related peptide, hypercalcemia of malignancy factor, parathyroid hormone-related protein (PTH-rP), glucagon-like peptide (GLP-1), pancreastatin, pancreatic peptide, peptide YY, PHM, secretin, vasoactive intestinal peptide (VIP), oxytocin, vasopressin (AVP), vasotocin, enkephalinamide, metorphinamide, alpha melanocyte stimulating hormone (alpha-MSH), atrial natriuretic factor (ANF), amylin, amyloid P component (SAP-1), corticotropin releasing hormone (CRH), growth hormone releasing factor (GHRH), luteinizing hormone-releasing hormone (LHRH), neuropeptide Y, substance K (neurokinin A), substance P, and thyrotropin releasing hormone (TRH).

Other desirable gene products include fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione β-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, β-glucosidase, pyruvate carboxylase, hepatic phosphorylase, phosphorylase kinase, glycine decarboxylase (also referred to as P-protein), H-protein, T-protein, Menkes disease copper-transporting ATPase, Wilson's disease copper-transporting ATPase, cytosine deaminase, hypoxanthine-guanine phosphoribosyltransferase, galactose-1-phosphate uridylyltransferase, galactokinase, UDP-galactose-4-epimerase, phenylalanine hydroxylase, glucocerbrosidase, sphingomyelinase, α-L-iduronidase, glucose-6-phosphate dehydrogenase, HSV thymidine kinase, human thymidine kinase, blood derivatives, growth factors, neurotransmitters or their precursors or synthetic enzymes, trophic factors (such as BDNF, CNTF, NGF, IGF, GMF, aFGF, bFGF, NT3, NT5, and the like), apolipoproteins (such as ApoAI, ApoAIV, ApoE, and the like), dystrophin or a minidystrophin, factor VII, factor VIII, factor IX, fibrin, fibrinogen, thrombin, cytosine deaminase, all or part of a natural or artificial immunoglobulin (Fab, ScFv, and the like), anti-thrombotic genes (e.g., COX-1, TFPI); genes involved in angiogenesis (e.g., VEGF, aFGF, bFGF, FGF-4, FGF-5, thrombospondin, BAI-1, GDAIF, or their receptors), MCC, and mouse or humanized monoclonal antibodies.

The protein can also be an antigenic peptide or polypeptide capable of generating an immune response. Examples include polynucleotides encoding antigens such as viral antigens, bacterial antigens, fungal antigens or parasitic antigens. Virus targets include picornavirus, coronavirus, togavirus, flavivirus, rhabdovirus, paramyxovirus, orthomyxovirus, bunyavirus, arenvirus, reovirus, retrovirus, papovavirus, parvovirus, herpesvirus, poxvirus, hepadnavirus, and spongiform virus. Parasite targets include trypanosomes, tapeworms, roundworms, and helminthes. Also, tumor markers, such as fetal antigen or prostate specific antigen, may be targeted in this manner.

Those of skill in the art are familiar with methods of electroporation. The electroporation may be, for example, flow electroporation or static electroporation. In one embodiment, the method of transfecting the cancer cells comprises use of an electroporation device as described in U.S. patent application Ser. No. 10/225,446, incorporated herein by reference. Methods and devices for electroporation are also described in, for example, published PCT Application Nos. WO 03/018751 and WO 2004/031353; U.S. patent application Ser. Nos. 10/781,440, 10/080,272, and 10/675,592; and U.S. Pat. Nos. 5,720,921, 6,074,605, 6,773,669, 6,090,617, 6,485,961, 6,617,154, 5,612,207, all of which are incorporated by reference.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A and 1B: AZA Effect on Viability of Electroporated K562 Cells Expressing GFP. K562 cells were transfected by electroporation (1 kV/cm, four 400 μs pulses) with 200 μg/ml of the plasmid CMV-eGFP or PGK-eGFP (phosphoglycerate kinase promoter regulated). Cells were cultured post-transfection in full medium with 0.0, 0.008, 0.04, 0.2, 1.0, or 5.0 μM AZA. Cell viability was assayed by PI exclusion using flow cytometry. As shown in FIGS. 1A and 1B, AZA had no effect on cell viability.

FIGS. 2A and 2B: AZA Effect on the Percentage of GFP+ K562 Cells. K562 cells were transfected by electroporation (1 kV/cm, four 400 μs pulses) with 200 μg/ml of the plasmid CMV-eGFP or PGK-eGFP. Cells were cultured post-transfection in full medium with 0.0, 0.008, 0.04, 0.2, 1.0, or 5.0 μM AZA. Cells were analyzed by flow cytometery. As shown in FIGS. 2A and 2B, at the plasmid concentration used in this study the percentage of GFP+ cells was not significantly dependent on AZA concentration.

FIGS. 3A and 3B: AZA Effect on GFP Expression Levels in Transfected K562 Cells. K562 cells were transfected by electroporation (1 kV/cm, four 400 μs pulses) with 200 μg/ml of the plasmid CMV-eGFP or PGK-eGFP. Cells were cultured post-transfection in full medium with 0.0, 0.008, 0.04, 0.2, 1.0, or 5.0 μM AZA. Cells were analyzed by flow cytometery. As shown in FIGS. 3A and 3B, AZA had a dose-dependent effect on the mean fluorescence intensity in cells transfected with CMV-eGFP or PGK-eGFP.

FIGS. 4A and 4B: AZA Effect on the Percentage of High-Level GFP Expressing K562 Cells. K562 cells were transfected by electroporation (1 kV/cm, four 400 μs pulses) with 200 μg/ml of the plasmid CMV-eGFP or PGK-eGFP. Cells were cultured post-transfection in full medium with 0.0, 0.008, 0.04, 0.2, 1.0, or 5.0 μM AZA. Cells were analyzed by flow cytometery. As shown in FIGS. 4A and 4B, AZA treatment had a dose-dependent effect on slowing down the decrease of the percentage of the high-level GFP expressing cells (mean>4×10³).

FIG. 5: AZA Effect on Viability of Electroporated K562 Cells Expressing hCD40L. K562 cells were transfected by electroporation (1 kV/cm, four 400 μs pulses) with 200 μg/ml of the plasmid CMV-hCD40L. Cells were cultured post-transfection in full medium with 0.0, 0.008, 0.04, 0.2, 1.0, or 5.0 μM AZA. The culture medium and AZA was changed every 24 hours. Cells were analyzed by flow cytometry. Cell viability was assayed by PI exclusion test. As shown in FIG. 5, AZA had no significant effect on cell viability.

FIG. 6: AZA Effect on the Percentage of CD40L+ K562 Cells. K562 cells were transfected by electroporation (1 kV/cm, four 400 μs pulses) with 200 μg/ml of the plasmid CMV-hCD40L. Cells were cultured post-transfection in full medium with 0.0, 0.008, 0.04, 0.2, 1.0, or 5.0 μM AZA. The culture medium and AZA was changed every 24 hours. Cells were analyzed by flow cytometry after immunostaining with monoclonal antibodies to hCD40L. As shown in FIG. 6, AZA had a dose-dependent effect on slowing down the decrease of the hCD40L expression level of the transfected cells.

FIG. 7: AZA Effect on hCD40L Expression Levels in Transfected K562 Cells. K562 cells were transfected by electroporation (1 kV/cm, four 400 μs pulses) with 200 μg/ml of the plasmid CMV-hCD40L. Cells were cultured post-transfection in complete medium with 0.0, 0.008, 0.04, 0.2, 1.0, or 5.0 μM AZA. The culture medium and AZA was changed every 24 hours. Cells were analyzed by flow cytometry after immunostaining with monoclonal antibodies to hCD40L. As shown in FIG. 7, AZA treatment had a dose-dependent effect on the rate of reduction of the decrease of the hCD40L expression level of transfected cells.

FIG. 8: AZA Effect on hIL-2 Expression Levels in K562 Cells. K562 cells were transfected by electroporation (1 kV/cm, four 400 μs pulses) with 200 μg/ml of the plasmid CMV-hIL-2. Cells were cultured post-transfection in full medium with 0.0, 0.008, 0.04, 0.2, 1.0, or 5.0 μM AZA. The secretion level of hIL-2 was analyzed by ELISA and presented as ng/24 h/1e5 input cells. As shown in FIG. 8, AZA had a dose-dependent effect on the rate of reduction of the decrease of the hIL-2 expression level of transfected cells.

FIG. 9: AZA Effect on mIL-12 Expression Levels in K562 Cells. K562 cells were transfected by electroporation (1 kV/cm, four 400 μs pulses) with 200 μg/ml of the plasmid CMV-mIL-12. Cells were cultured post-transfection in full medium with 0.0, 0.008, 0.04, 0.2, 1.0, or 5.0 μM AZA. The secretion level of mIL-12 was analyzed by ELISA and presented as ng/24 h/1e5 input cells. As shown in FIG. 9, AZA had a dose-dependent effect on the rate of reduction of the decrease of the expression level of transfected cells.

FIG. 10: AZA Effect on Viability of Electroporated 293T Cells Expressing GFP. 293T cells were transfected by electroporation (1 kV/cm, four 400 μs pulses) with 200 μg/ml of the plasmid CMV-eGFP. Cells were cultured post-transfection in full medium with 0.0, 0.008, 0.04, 0.2, 1.0, or 5.0 μM AZA. The culture medium and AZA was changed every 24 hours. Cell viability was assayed by PI exclusion using flow cytometry. As shown in FIG. 10, AZA had no effect on cell viability.

FIG. 11: AZA Effect on the Percentage of GFP+ 293T Cells. 293T cells were transfected by electroporation (1 kV/cm, four 400 μs pulses) with 200 μg/ml of the plasmid CMV-eGFP. Cells were cultured post-transfection in full medium with 0.0, 0.008, 0.04, 0.2, 1.0, or 5.0 μM AZA. The culture medium and AZA was changed every 24 hours. Cells were analyzed by flow cytometry. As shown in FIG. 11, the percentage of GFP+ cells was not significantly dependent on AZA concentration.

FIG. 12: AZA Effect on GFP Expression Levels in Transfected 293T Cells. 293T cells were transfected by electroporation (1 kV/cm, four 400 μs pulses) with 200 μg/ml of the plasmid CMV-eGFP. Cells were cultured post-transfection in full medium with 0.0, 0.008, 0.04, 0.2, 1.0, or 5.0 μM AZA. The culture medium and AZA was changed every 24 hours. Cells were analyzed by flow cytometry. As shown in FIG. 12, AZA had a dose-dependent effect on the rate of reduction of the decrease of the GFP expression level in transfected cells.

FIG. 13: AZA Effect on the Percentage of High-Level GFP Expressing 293T Cells. 293T cells were transfected by electroporation (1 kV/cm, four 400 μs pulses) with 200 μg/ml of the plasmid CMV-eGFP. Cells were cultured post-transfection in full medium with 0.0, 0.008, 0.04, 0.2, 1.0, or 5.0 μM AZA. The culture medium and AZA was changed every 24 hours. Cells were analyzed by flow cytometry. As shown in FIG. 13, AZA had a dose-dependent effect on the rate of reduction of the decrease of the percentage of the high-level GFP expressing cells (mean>4×10³).

FIG. 14: AZA Effect on mIL-12 Expression Level in Transfected 293T Cells. 293T cells were transfected by electroporation (1 kV/cm, four 400 μs pulses) with 200 μg/ml of the plasmid CMV-mIL-12. Cells were cultured post-transfection in full medium with 0.0, 0.008, 0.04, 0.2, 1.0, or 5.0 μM AZA. The secretion level of mIL-12 was analyzed by ELISA and presented as ng/24 h. As shown in FIG. 15, AZA had a dose-dependent effect on the rate of reduction of the decrease of the mIL-12 expression level of transfected cells.

FIG. 15: AZA Effect on Cell Proliferation of Transfected K562 Cells. K562 cells were transfected by electroporation (1 kV/cm, four 400 μs pulses) with 200 μg/ml of the plasmid CMV-eGFP. Cells were cultured post-transfection in full medium with 0.0, 0.008, 0.04, 0.2, 1.0, or 5.0 μM AZA. The cell concentration was analyzed by hemacytometer. As shown in FIG. 16, AZA retarded cell growth in a dose-dependent manner.

FIG. 16: AZA Effect on the Morphology of Transfected K562 Cells. K562 cells were transfected by electroporation (1 kV/cm, four 400 μs pulses) with 200 μg/ml of the plasmid CMV-eGFP. Cells were cultured for 7 days post-transfection in full medium with the indicated AZA concentrations.

FIG. 17: Effect of Pre-Transfection AZA Treatment on Cell Viability. Prior to transfection, K562 cells were cultured overnight in complete medium with the indicated AZA concentrations. The cells were transfected by electroporation (1 kV/cm, four 400 μs pulses) with 200 μg/ml plasmid concentration. Following transfection, each transfected cell sample was divided in to two parts and cultured in medium either without additional AZA (solid symbols) or with 5 μM AZA (empty symbols). Cell viability was assayed by PI exclusion using flow cytometry at 4.5, 22 and 48 hours post transfection.

FIG. 18: Effect of Pre-Transfection AZA Treatment on the Percentage of GFP+ Cells. Prior to transfection, K562 cells were cultured overnight in complete medium with the indicated AZA concentrations. The cells were transfected by electroporation (1 kV/cm, four 400 μs pulses) with 200 μg/ml plasmid concentration. Following transfection, each transfected cell sample was divided in to two parts and cultured in medium either without additional AZA (solid symbols) or with 5 μM AZA (empty symbols). Transfection efficiency (GFP+ cells) was assayed by flow cytometric analysis at 4.5, 22 and 48 hours post transfection.

FIG. 19: Effect of Pre-Transfection AZA Treatment on Transgene Expression. Prior to transfection, K562 cells were cultured overnight in complete medium with the indicated AZA concentrations. The cells were transfected by electroporation (1 kV/cm, four 400 μs pulses) with 200 μg/ml plasmid concentration. Following transfection, each transfected cell sample was divided in to two parts and cultured in medium either without additional AZA (solid symbols) or with 5 μM AZA (empty symbols). Mean fluorescence intensity (MFI) was assayed by flow cytometric analysis at 4.5, 22 and 48 hours post transfection.

FIG. 20: Effect of Pre-Transfection AZA Treatment on Transgene Expression at 4.5 Hours Post-Transfection. Prior to transfection, K562 cells were cultured overnight in complete medium with the indicated AZA concentrations. The cells were transfected by electroporation (1 kV/cm, four 400 μs pulses) with 200 μg/ml plasmid concentration. Following transfection, each transfected cell sample was divided in to two parts and cultured in medium either without additional AZA (solid symbols) or with 5 μM AZA (empty symbols). Mean fluorescence intensity (MFI) was assayed by flow cytometric analysis at 4.5 hours post transfection.

FIG. 21: AZA Treatment Improves Viral Vector Production. Three plasmids coding for a transgene (eGFP), lentiviral viral gag/pol, and the viral envelop protein VSVG were co-transfected by electroporation into 293T cells with DNA ratios I, II, III and IV (eGFP: gag/pol: VSVG ratios of 50:20:0.625, 50:20:1.25, 50:20:2.5 and 50:20:5, respectively) to produce viral particles. Each transfected sample was divided into two parts following transfection and grown in culture medium with 5 μM AZA (+AZA) or without AZA (−AZA). Virus-containing supernatant was collected at 24 hours, 48 hours, and 72 hours post transfection and were titered using D17 cells.

### DESCRIPTION OF ILLUSTRATIVE EMBODIMENTS

A. Electroporation

The present invention provides methods for enhancing transgene expression in cells transfected by electroporation. As used herein, “electroporation” refers to application of an electrical current or electrical field to a cell to facilitate entry of a nucleic acid molecule or other molecule into the cell. One of skill in the art would understand that any method and technique of electroporation is contemplated by the present invention. However, in certain embodiments of the invention, electroporation may be carried out as described in U.S. patent application Ser. No. 10/225,446, filed Aug. 21, 2002, the entire disclosure of which is specifically incorporated herein by reference.

In other embodiments of the invention, electroporation may be carried out as described in U.S. Pat. No. 5,612,207 (specifically incorporated herein by reference), U.S. Pat. No. 5,720,921 (specifically incorporated herein by reference), U.S. Pat. No. 6,074,605 (specifically incorporated herein by reference); U.S. Pat. No. 6,090,617 (specifically incorporated herein by reference); and U.S. Pat. No. 6,485,961 (specifically incorporated herein by reference).

Other methods and devices for electroporation that may be used in the context of the present invention are also described in, for example, published PCT Application Nos. WO 03/018751 and WO 2004/031353; U.S. patent application Ser. Nos. 10/781,440, 10/080,272, and 10/675,592; and U.S. Pat. Nos. 6,773,669, 6,090,617, 6,617,154, all of which are incorporated by reference.

Electroporation has been described as a means to introduce nonpermeant molecules into living cells (reviewed in Mir, 2000). At the level of the entire cell, the consequences of cell exposure to the electric pulses are not completely understood. In the presence of the external electric field, a change in the transmembrane potential difference is believed to be generated (Neumann et al., 1999; Weaver and Chizmadzhev, 1996; Kakorin et al., 1996). It superimposes upon the resting transmembrane potential difference and it may be calculated from the Maxwell's equations, providing a few approximations are made (very reduced thickness of the cell membrane, null membrane conductivity, etc.) (Mir, 2000). These changes in the transmembrane potential difference have been experimentally observed (Hibino et al., 1993; Gabriel and Teissie, 1999). Analytically, the effects of the exposure of cells to electric pulses are well described in the case of isolated cells in suspension (Kotnik et al., 1998).

At the molecular level of analysis, the explanation of the phenomena occurring at the cell membrane level is hypothetical. It is assumed that above a threshold value of the net transmembrane potential, the changes occurring in membrane structure will be enough as to render that membrane permeable to otherwise nonpermeant molecules of given physicochemical characteristics (molecular mass, radius, etc.) (Mir, 2000).

DNA electroporation was originally described using simple generators that produce exponentially decaying pulses. Square-wave electric pulse generators were later developed that allowed specification of the various electric parameters (pulse intensity, pulse length, number of pulses) (Rols and Teissié, 1990). The selection of parameters is dependent on the cell type being electroporated and physical characteristics of the molecules that are to be taken up by the cell.

As generally practiced in vitro, electroporation is carried out in small (less than 0.5 milliliters) cuvette-like chambers containing a pair of electrodes with motionless cells and fluid (“static” EP). The volume of chambers for static EP determines the maximal amount of cells that can be conveniently electroporated. Static EP devices electroporate enough cells for many laboratory research applications but not enough for either industrial applications or cell-based therapy. Theoretically, large volumes could be electroporated by pooling large numbers of small batches from static electroporation. This, however, would be time consuming or require simultaneous use of multiple electroporation apparatuses, which would be costly and exacerbate problems of reproducibility and quality assurance.

Flow EP and streaming EP are two technologies that enable the processing of large volumes of cells. Thus, flow EP and streaming EP are better suited to industrial applications and cell-based therapy than is static EP. The application of an electric field (EF) to cells in conventional “flow” EP is typically the same as for static EP: a pulse of electrical energy is applied at certain time intervals that are long when compared to the time duration of each individual pulse. In conventional flow EP, computer-controlled electronic switches typically close repeatedly to deliver distinct HV pulses to a new batch of cells once a prior batch of cells are displaced by a pump out of the space between electrodes. In some respects, therefore, conventional flow EP processes are similar to static EP—in the way that EF is applied to the electrodes and to the sample. The two processes differ, however, in the way samples are handled—one is static while the other is characterized by batch-wise flowing.

In both static and conventional flow EP methods, the transient nature of the electric field experienced by the sample being electroporated is the result of electronic control over the magnitude and duration of one or more voltage pulses applied to the electrodes. In the case of flow EP, the flow rate of cells between the electrodes must be coordinated with the rate of high-voltage pulse application.

With “streaming” electroporation, it is the sample streaming relative to an electric field that primarily determines the exposure of the sample to the electric field that effects electroporation. This, of course, is in contrast to conventional techniques in which the duration of an electrical pulse (or pulses) applied to electrodes primarily determines the exposure of the sample to an electric field. In other words, in streaming EP, the rate of relative motion between an electric field and a sample can be used to achieve electroporation instead of signal pulsing applied to the electrodes. Streaming EP can utilize signal pulsing, although that pulsing no longer acts as the primary mechanism for achieving electroporation.

In streaming EP, biological cells are effectively “pulsed” by their defined movement across electrical field lines. Each cell moves across electric field lines and is exposed to an electric field for the period of time it spends between the electrodes (which is analogous to a pulse width in a typical application). The field quickly increases as the cells approach the space between the electrodes, reaches its maximum and decreases as the cells leave this space. The cell exposure time equals the ratio of electrode length in the direction of flow to the linear velocity of cell movement.

B. DNA Methylation

The present invention provides a method for enhancing transgene expression in a cell. This method comprises contacting the cell with a methyltransferase inhibitor, wherein the expression of the transgene is enhanced as compared to the expression of the transgene in a similar cell that was not contacted with a methyltransferase inhibitor.

DNA methylation is one mechanism used by cells to regulate endogenous gene expression. Gene expression is generally associated with demethylation or hypomethylation. Some genes, however, can be expressed even when they are extensively methylated. The methylation state of a gene can vary temporally and spatially. For example, the methylation state of a particular gene can change over time as it is switched “on” or “off” during the course of development. As another example, the methylation state of a particular gene can vary among tissues. A gene may be methylated in a tissue where it is not expressed, and undermethylated in a tissue where it is expressed.

Most of the methyl groups are found in CG “doublets” in the DNA sequence. The methylation of cytosine (C) to form 5-methylcytosine occurs enzymatically after DNA synthesis. The distribution of methyl groups can be examined using restriction enzymes that cleave target sites containing the CG doublet. For example, the enzyme MspI cleaves all CCGG sequences whether they are methylated or not, whereas the enzyme HpaII cleaves only nonmethylated CCGG sequences.

There are a number of inhibitors of DNA methylation known in the art. Examples include 5′-azacytidine, 5′-aza-2′-deoxycytidine, L-ethionine, and 2-pyrimidone-1-β-D-riboside-1-(β-D-ribofuranosyl)-1,2-dihydropyrimidin-2-one (Zebularine). DNA methylation can also be inhibited by inhibitory nucleic acids (e.g., antisense oligonucleotides; siRNA) targeting DNA methyltransferease genes such as human DNA methyltransferase I (Dnmt-1). It is generally accepted that RNA inhibition (RNAi) acts post-transcriptionally, targeting RNA transcripts for degradation. siRNAs, for example, are designed so that they are specific and effective in suppressing the expression of the genes of interest. Thus, to inhibit DNA methylation, siRNAs would be designed to target DNA methyltransferase enzymes. Other proteins associated with the silencing of methylated DNA could also be targeted. In some embodiments it may be desirable to target two or more different proteins involved in DNA methylation and/or the silencing of methylated DNA. Typically, siRNA sequences of about 21 to 23 nucleotides in length are most effective. This length reflects the lengths of digestion products resulting from the processing of much longer RNAs. The making of siRNAs has been mainly through direct chemical synthesis; through processing of longer, double stranded RNAs through exposure to Drosophila embryo lysates; or through an in vitro system derived from S2 cells.

5′-azacytidine and 5′-aza-2′-deoxycytidine are well-known methyltransferase inhibitors. They are analogs of cytidine in which a nitrogen atom replaces the carbon at the 5′ position of the pyrimidine ring. There are at least two reported mechanism by which it is thought that 5′-azacytidine and 5′-aza-2′-deoxycytidine inhibit DNA methylation. First, the 5′ nitrogen atom cannot be methylated; therefore, the incorporation of 5′-azacytidine or 5′-aza-2′-deoxycytidine in the DNA results in hypomethylation of the DNA. Furthermore, 5′-azacytidine and 5′-aza-2′-deoxycytidine are believed to covalently bind DNA methyltransferease, thus depleting available enzyme and causing demethylation of genomic DNA.

As demonstrated herein, 5′-aza-2′-deoxycytidine successfully increased and prolonged the electroporation-mediated transgene expression for different cell lines with different growth characteristics (K562 and 293T); for plasmids with different promoters (the viral CMV promoter and the mammalian PGK promoter); and for different expressed proteins (cytosolic, membrane, and secreted proteins).

For the proteins secreted by transfected 293T cells, there was a 5-fold increase of the total secreted protein for 5 μM AZA-treated cells as compared to non-AZA treated cells over the total 5 day period. If only comparing the data on day 5, there was a 10-fold increase of the secreted protein by AZA-treated 293T cells over that by non-AZA-treated 293T cells. The results suggest that AZA has a universal effect in increasing and prolonging the electroporation-mediated transgene expression, demonstrating the usefulness of methyltransferase inhibitors for protein and virus production.

High concentrations of methyltransferase inhibitors can be toxic to cells. In the studies described herein, the cell number with 5 μM AZA treatment in the experiments of secretion of hIL-2 and mIL-12 was lower than that without AZA treatment. However, 5 μM AZA treatment also resulted in the highest improvement of transgene expression. A person of ordinary skill in the art would be able to determine the appropriate concentration of a methyltransferase inhibitor to use depending on the desired effect to be achieved. For example, if both increasing cell number and enhancing transgene expression are desired then the methyltransferase inhibitor would be used at a concentration that causes low toxicity or no toxicity to the cell. A straightforward method for assessing toxicity is to count the number of cells using a hemocytometer.

The cells are preferably exposed to the methyltransferase inhibitor before and after electroporation. However, the cells may be exposed to the methyltransferase inhibitor during electroporation. Typically, the methyltransferase inhibitor will be added to the culture medium. The concentration of methyltransferase inhibitor will vary depending on the cell type, the particular methyltransferase inhibitor, and the level of expression desired. For example the concentration may be 0.005, 0.008, 0.01, 0.04, 0.08, 0.1, 0.2, 0.25, 0.4, 0.5, 0.75, 1.0, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 12, 14, 16, 18, 20, or 25 μM. It is also envisioned that the methyltransferase inhibitor may be incorporated into the vector prior to transfection into the cell.

C. Nucleic Acid-Based Expression Systems

1. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Goodbourn and Maniatis et al., 1988 and Ausubel et al., 1996, both incorporated herein by reference).

The term “expression vector” refers to any type of genetic construct comprising a nucleic acid coding for an RNA capable of being transcribed and then translated into a protein, polypeptide, or peptide. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles, such as mitochondria, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 2001, incorporated herein by reference). The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 1 lists non-limiting examples of elements/promoters that may be employed, in the context of the present invention, to regulate the expression of a RNA. Table 2 provides non-limiting examples of inducible elements, which are regions of a nucleic acid sequence that can be activated in response to a specific stimulus.

TABLE 1
Promoter and/or Enhancer

| Promoter/Enhancer | References |
|---|---|
| Immunoglobulin Heavy Chain | Banerji et al., 1983; Gilles et al., 1983; |
| | Grosschedl et al., 1985; Atchinson et al., |
| | 1986, 1987; Imler et al., 1987; Weinberger |
| | et al., 1984; Kiledjian et al., 1988; |
| | Porton et al.; 1990 |
| Immunoglobulin Light Chain | Queen et al., 1983; Picard et al., 1984 |
| T-Cell Receptor | Luria et al., 1987; Winoto et al., 1989; |
| | Redondo et al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase | Jaynes et al., 1988; Horlick et al., 1989; |
| (MCK) | Johnson et al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, |
| | 1990 |
| α-Fetoprotein | Godbout et al., 1988; Campere et al., 1989 |
| γ-Globin | Bodine et al., 1987; Perez-Stable et al., |
| | 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsch et al., 1990 |
| Molecule (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A | Edbrooke et al., 1989 |
| (SAA) | |
| Troponin I (TN I) | Yutzey et al., 1989 |
| Platelet-Derived Growth | Pech et al., 1989 |
| Factor (PDGF) | |
| Duchenne Muscular | Klamut et al., 1990 |
| Dystrophy | |
| SV40 | Banerji et al., 1981; Moreau et al., 1981; |
| | Sleigh et al., 1985; Firak et al., 1986; Herr |
| | et al., 1986; Imbra et al., 1986; Kadesch et |
| | al., 1986; Wang et al., 1986; Ondek et al., |
| | 1987; Kuhl et al., 1987; Schaffner et al., |
| | 1988 |
| Polyoma | Swartzendruber et al., 1975; Vasseur et |
| | al., 1980; Katinka et al., 1980, 1981; |
| | Tyndell et al., 1981; Dandolo et al., 1983; |
| | de Villiers et al., 1984; Hen et al., 1986; |
| | Satake et al., 1988; Campbell and/or |
| | Villarreal, 1988 |
| Retroviruses | Kriegler et al., 1982, 1983; Levinson et |
| | al., 1982; Kriegler et al., 1983, 1984a, b, |
| | 1988; Bosze et al., 1986; Miksicek et al., |
| | 1986; Celander et al., 1987; Thiesen et al., |
| | 1988; Celander et al., 1988; Choi et al., |
| | 1988; Reisman et al., 1989 |
| Papilloma Virus | Campo et al, 1983; Lusky et al., 1983; |
| | Spandidos and/or Wilkie, 1983; Spalholz |
| | et al., 1985; Lusky et al., 1986; Cripe |
| | et al., 1987; Gloss et al., 1987; Hirochika |
| | et al., 1987; Stephens et al., 1987 |
| Hepatitis B Virus | Bulla et al., 1986; Jameel et al., 1986; |
| | Shaul et al., 1987; Spandau et al., 1988; |
| | Vannice et al., 1988 |
| Human Immunodeficiency | Muesing et al., 1987; Hauber et al., 1988; |
| Virus | Jakobovits et al., 1988; Feng et al., 1988; |
| | Takebe et al., 1988; Rosen et al., 1988; |
| | Berkhout et al., 1989; Laspia et al., 1989; |
| | Sharp et al., 1989; Braddock et al., 1989 |
| Cytomegalovirus (CMV) | Weber et al., 1984; Boshart et al., 1985; |
| | Foecking et al., 1986 |
| Gibbon Ape Leukemia Virus | Holbrook et al., 1987; Quinn et al., 1989 |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart et |
| | | al., 1985; Imagawa et al., |
| | | 1987, Karin et al., 1987; |
| | | Angel et al., 1987b; |
| | | McNeall et al., 1989 |
| MMTV (mouse | Glucocorticoids | Huang et al., 1981; Lee et |
| mammary tumor | | al., 1981; Majors et al., |
| virus) | | 1983; Chandler et al., 1983; |
| | | Lee et al., 1984; Ponta et |
| | | al., 1985; Sakai et al., 1988 |
| β-Interferon | Poly(rI)x | Tavernier et al., 1983 |
| | Poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor α | | |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Non-limiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (Wu et al., 1997), and human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

The use of internal ribosome entry sites (IRES) elements may be used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al, 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to be more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

i. Plasmid Vectors

In certain embodiments, a plasmid vector is contemplated for use to transform a cell. In general, plasmid vectors containing replicon and control sequences which are derived from species compatible with the cell are used in connection with these cells. The vector ordinarily carries a replication site, as well as marking sequences which are capable of providing phenotypic selection in transformed cells.

D. Host Cells and Expression Systems

1. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells may be derived from prokaryotes or eukaryotes, including bacteria cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials. An appropriate host can be determined by one of skill in the art based on the vector backbone and the desired result. Examples of mammalian cells that can be used in the context of the present invention include, but are not limited to, human embryonic kidney cells, K562 cells, 293T cells, Vero cells, CHO cells, HeLa cells, W138, BHK, COS-7, HepG2, 3T3, RIN, dendritic cells, T cell, B cells, and MDCK cells or any eukaryotic cells for which tissue culture techniques are established.

In certain embodiments, it may be useful to employ selection systems that preclude growth of undesirable cells. This may be accomplished by virtue of permanently transforming a cell line with a selectable marker or by transducing or infecting a cell line with a vector that encodes a selectable marker. In either situation, culture of the transformed/transduced cell with an appropriate drug or selective compound will result in the enhancement, in the cell population, of those cells carrying the marker.

Examples of markers include, but are not limited to, HSV thymidine kinase, hypoxanthine-guanine phosphoribosyltransferase and adenine phosphoribosyltransferase genes, in tk−, hgprt− or aprt− cells, respectively. Also, anti-metabolite resistance can be used as the basis of selection for dhfr, that confers resistance to methotrexate; gpt, that confers resistance to mycophenolic acid; neo, that confers resistance to the aminoglycoside G418; and hygro, that confers resistance to hygromycin.

2. Cell Culture Systems

In eukaryotic cell culture systems, the culture of the cells is generally under conditions of controlled pH, temperature, humidity, osmolarity, ion concentrations, and exchange of gases. Regarding the latter, oxygen and carbon dioxide are of particular importance to the culturing of cells. In a typical eukaryotic cell culture system, an incubator is provided in which carbon dioxide is infused to maintain an atmosphere of about 5% carbon dioxide within the incubator. The carbon dioxide interacts with the tissue culture medium, particularly its buffering system, in maintaining the pH near physiologic levels.

In addition to carbon dioxide, the culturing of cells is dependent upon the ability to supply to the cells a sufficient amount of oxygen necessary for cell respiration and metabolic function. Methods to increase oxygen concentration to the cultured cells include mechanical stirring, medium perfusion or aeration, increasing the partial pressure of oxygen, and/or increasing the atmospheric pressure.

Conventional cell culture containers comprise tissue culture flasks, tissue culture bottles, and tissue culture plates. Gas exchange between the incubator atmosphere and a tissue culture plate generally involves a loosely fitting cover which overhangs the plate. Similarly, for a tissue culture flasks or bottle, a loosely fitting cap excludes particulate contaminants from entering the chamber of the flask or bottle, but allows gas exchange between the incubator atmosphere and the atmosphere within the flask or bottle. Caps with a gas permeable membrane or filter are also available, thereby allowing for gas exchange with a tightly fitting cap.

As used herein, “media” and “medium” refers to any substance which can facilitate growth of cells. One of skill in the art would be familiar with the wide range of types of media available which can be used in cell culture systems. In certain embodiments of the present invention, the host cells are grown in media that is serum-free media. In other embodiments of the present invention, the host cells are grown in media that is protein-free media. One of skill in the art would understand that various components and agents can be added to the media to facilitate and control cell growth. For example, the glucose concentration of the media can be maintained at a certain level.

Mammalian cells can be propagated in vitro in two modes: as non-anchorage dependent cells growing freely in suspension throughout the bulk of the culture; or as anchorage-dependent cells requiring attachment to a solid substrate for their propagation (i.e., a monolayer type of cell growth). Traditionally, anchorage-dependent cell cultures are propagated on the bottom of small glass or plastic vessels. A number of techniques have been proposed that offer large accessible surfaces for cell growth: the roller bottle system, the stack plates propagator, the spiral film bottle, the hollow fiber system, the packed bed, the plate exchanger system, and the membrane tubing reel. The roller bottle system is a commonly used process for large scale anchorage-dependent cell production. Fully automated robots are available that can handle thousands of roller bottles per day, thus eliminating the risk of contamination and inconsistency associated with the otherwise required intense human handling. With frequent media changes, roller bottle cultures can achieve cell densities of close to 0.5×10⁶ cells/cm² (corresponding to approximately 10⁹ cells/bottle or almost 10⁷ cells/ml of culture media).

In an effort to overcome the shortcomings of the traditional anchorage-dependent culture processes, van Wezel (1967) developed the concept of microcarrier culturing systems. In this system, cells are propagated on the surface of small solid particles suspended in the growth medium by slow agitation. Cells attach to the microcarriers and grow gradually to confluency on the microcarrier surface. In fact, this large scale culture system upgrades the attachment dependent culture from a single disc process to a unit process in which both monolayer and suspension culture have been brought together. Thus, combining the necessary surface for a cell to grow with the advantages of the homogeneous suspension culture increases production.

The advantages of microcarrier cultures over most other anchorage-dependent, large-scale cultivation methods are several fold. First, microcarrier cultures offer a high surface-to-volume ratio (variable by changing the carrier concentration), which leads to high cell density yields and a potential for obtaining highly concentrated cell products. Cell yields are up to 1-2×10⁷ cells/ml when cultures are propagated in a perfused reactor mode. Second, cells can be propagated in one unit process vessels instead of using many small low-productivity vessels (i.e., flasks or dishes). Third, the well-mixed and homogeneous microcarrier suspension culture makes it possible to monitor and control environmental conditions (e.g., pH, pO2, and concentration of medium components), thus leading to more reproducible cell propagation and product recovery. Fourth, it is possible to take a representative sample for microscopic observation, chemical testing, or enumeration. Fifth, since microcarriers settle out of suspension quickly, use of a fed-batch process or harvesting of cells can be done relatively easily. Sixth, the mode of the anchorage-dependent culture propagation on the microcarriers makes it possible to use this system for other cellular manipulations, such as: cell transfer without the use of proteolytic enzymes; cocultivation of cells; transplantation into animals; and perfusion of the culture using decanters, columns, fluidized beds, or hollow fibers for microcarrier retainment. Seventh, microcarrier cultures are relatively easily scaled up using conventional equipment used for cultivation of microbial and animal cells in suspension.

One method which has shown to be particularly useful for culturing mammalian cells is microencapsulation. The mammalian cells are retained inside a semipermeable hydrogel membrane. A porous membrane is formed around the cells permitting the exchange of nutrients, gases, and metabolic products with the bulk medium surrounding the capsule. Several methods have been developed that are gentle, rapid and non-toxic and where the resulting membrane is sufficiently porous and strong to sustain the growing cell mass throughout the term of the culture. These methods are all based on soluble alginate gelled by droplet contact with a calcium-containing solution. Lim (1982, U.S. Pat. No. 4,352,883, incorporated herein by reference), describes cells concentrated in an approximately 1% solution of sodium alginate that are forced through a small orifice, forming droplets, and breaking free into an approximately 1% calcium chloride solution. The droplets are then cast in a layer of polyamino acid that ionically bonds to the surface alginate. Finally the alginate is reliquified by treating the droplet in a chelating agent to remove the calcium ions. Other methods use cells in a calcium solution to be dropped into an alginate solution, thus creating a hollow alginate sphere. A similar approach involves cells in a chitosan solution dropped into alginate, also creating hollow spheres.

Microencapsulated cells are easily propagated in stirred tank reactors and, with bead sizes in the range of 150-1500 μm in diameter, are easily retained in a perfused reactor using a fine-meshed screen. The ratio of capsule volume to total media volume can be maintained from as dense as 1:2 to 1:10. With intracapsular cell densities of up to 10⁸, the effective cell density in the culture is 1-5×10⁷. The advantages of microencapsulation include: the protection from the deleterious effects of shear stresses that occur from sparging and agitation, the ability to easily retain beads for the purpose of using perfused systems, the ability to scale up the process, and the ability to use the beads for implantation.

Perfusion refers to continuous flow at a steady rate, through or over a population of cells of a physiological nutrient solution. It implies the retention of the cells within the culture unit as opposed to continuous-flow culture, which washes the cells out with the withdrawn media (e.g., chemostat). The technique was initiated to mimic the cells milieu in vivo where cells are continuously supplied with blood, lymph, or other body fluids. Without perfusion, cells in culture go through alternating phases of being fed and starved, thus limiting full expression of their growth and metabolic potential.

The current use of perfused culture is in response to the challenge of growing cells at high densities (e.g., 0.1-5×10⁸ cells/ml). In order to increase densities beyond 2-4×10⁶ cells/ml, the medium has to be constantly replaced with a fresh supply in order to make up for nutritional deficiencies and to remove toxic products. Perfusion allows for a far better control of the culture environment (pH, pO2, nutrient levels, etc.) and is a means of significantly increasing the utilization of the surface area within a culture for cell attachment.

The development of a perfused packed-bed reactor using a bed matrix of a non-woven fabric has provided a means for maintaining a perfusion culture at densities exceeding 10⁸ cells/ml of the bed volume (CelliGen™, New Brunswick Scientific, Edison, N.J.; Wang et al., 1992; Wang et al., 1993; Wang et al., 1994). Briefly described, this reactor comprises an improved reactor for culturing of both anchorage- and non-anchorage-dependent cells. The reactor is designed as a packed bed with a means to provide internal recirculation. Preferably, a fiber matrix carrier is placed in a basket within the reactor vessel. A top and bottom portion of the basket has holes, allowing the medium to flow through the basket. A specially designed impeller provides recirculation of the medium through the space occupied by the fiber matrix for assuring a uniform supply of nutrient and the removal of wastes. This simultaneously assures that a negligible amount of the total cell mass is suspended in the medium. The combination of the basket and the recirculation also provides a bubble-free flow of oxygenated medium through the fiber matrix. The fiber matrix is a non-woven fabric having a “pore” diameter of from 10 μm to 100 μm, providing for a high internal volume with pore volumes corresponding to 1 to 20 times the volumes of individual cells.

The perfused packed-bed reactor offers several advantages. With a fiber matrix carrier, the cells are protected against mechanical stress from agitation and foaming. The free medium flow through the basket provides the cells with optimum regulated levels of oxygen, pH, and nutrients. Products can be continuously removed from the culture and the harvested products are free of cells and can be produced in low-protein medium, which facilitates subsequent purification steps. Also, the design of this reactor system makes it possible to scale up the reactor. This technology is explained in detail in WO 94/17178 (Aug. 4, 1994, Freedman et al.), which is hereby incorporated by reference in its entirety.

The Cellcube™ (Corning-Costar) module provides a large styrenic surface area for the immobilization and growth of substrate attached cells. It is an integrally encapsulated sterile single-use device that has a series of parallel culture plates joined to create thin sealed laminar flow spaces between adjacent plates.

The Cellcube™ module has inlet and outlet ports that are diagonally opposite each other and help regulate the flow of media. During the first few days of growth the culture is generally satisfied by the media contained within the system after initial seeding. The amount of time between the initial seeding and the start of the media perfusion is dependent on the density of cells in the seeding inoculum and the cell growth rate. The measurement of nutrient concentration in the circulating media is a good indicator of the status of the culture. When establishing a procedure it may be necessary to monitor the nutrients composition at a variety of different perfusion rates to determine the most economical and productive operating parameters.

Cells within the system reach a higher density of solution (cells/ml) than in traditional culture systems. Many typically used basal media are designed to support 1-2×10⁶ cells/ml/day. A typical Cellcube™, run with an 85,000 cm² surface, contains approximately 6 L media within the module. The cell density often exceeds 10⁷ cells/mL in the culture vessel. At confluence, 2-4 reactor volumes of media are required per day.

The timing and parameters of the production phase of cultures depends on the type and use of a particular cell line. Many cultures require a different media for production than is required for the growth phase of the culture. The transition from one phase to the other will likely require multiple washing steps in traditional cultures. However, the Cellcube™ system employs a perfusion system. One of the benefits of such a system is the ability to provide a gentle transition between various operating phases. The perfusion system negates the need for traditional wash steps that seek to remove serum components in a growth medium.

Suspension culture systems are particularly suitable for use in the present invention, as they reduce the amount of handling required to electroporate and culture the cells. For example, cells growing in a bioreactor can be transferred to an electroporation chamber for transfection and then to a bioreactor for further culture. The movement of the cells through the system may be automated. Furthermore, coupling the cell culture system to a flow electroporation system or a streaming electroporation system would allow rapid, large-scale processing.

Two suspension culture bioreactor designs are widely used in the industry due to their simplicity and robustness of operation—the stirred bioreactor and the airlift bioreactor. Agitation of the culture medium may also be achieved by axial rocking of a planar platform to induce wave motions inside of the bioreactor. The stirred bioreactor design has successfully been used on a scale of 8000 liter capacity for the production of interferon (Phillips et al., 1985; Mizrahi, 1983). Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift bioreactor, also initially described for microbial fermentation and later adapted for mammalian culture, relies on a gas stream to both mix and oxygenate the culture. The gas stream enters a riser section of the bioreactor and drives circulation. Gas disengages at the culture surface, causing denser liquid free of gas bubbles to travel downward in the downcomer section of the bioreactor. The main advantage of this design is the simplicity and lack of need for mechanical mixing. Typically, the height-to-diameter ratio is 10:1. The airlift reactor scales up relatively easily, has good mass transfer of gasses and generates relatively low shear forces.

Most large-scale suspension cultures are operated as batch or fed-batch processes because they are the most straightforward to operate and scale up. However, continuous processes based on chemostat or perfusion principles are available.

A batch process is a closed system in which a typical growth profile is seen. A lag phase is followed by exponential, stationary and decline phases. In such a system, the environment is continuously changing as nutrients are depleted and metabolites accumulate. This makes analysis of factors influencing cell growth and productivity, and hence optimization of the process, a complex task. Productivity of a batch process may be increased by controlled feeding of key nutrients to prolong the growth cycle. Such a fed-batch process is still a closed system because cells, products, and waste products are not removed.

In what is still a closed system, perfusion of fresh medium through the culture can be achieved by retaining the cells with a variety of devices (e.g. fine mesh spin filter, hollow fiber or flat plate membrane filters, settling tubes). A true open system and the simplest perfusion process is the chemostat in which there is an inflow of medium and an outflow of cells and products. Culture medium is fed to the reactor at a predetermined and constant rate which maintains the dilution rate of the culture at a value less than the maximum specific growth rate of the cells (to prevent washout of the cell mass from the reactor). Culture fluid containing cells and cell products and byproducts is removed at the same rate. One of skill in the art would be familiar with the various types of filters that can be used for perfusion of media, and the various methods that can be employed for attaching the filter to the bioreactor and incorporating it into the cell growth process.

D. Protein, Virus, and Transgenic Cell Production

As mentioned above, the present invention is directed to methods of enhancing electroporation-mediated transgene expression. The methods described herein provide increased and prolonged expression of transgenic proteins. Accordingly, the present invention is well suited for the production of proteins, viruses, and transgenic cells. Furthermore, the methods are readily compatible with large-volume cell culture systems and high-throughput electroporation systems. Consequently, the present invention is well suited for the large-scale production of proteins, viruses, and transgenic cells.

Therapeutic proteins, as well as proteins having other research, commercial, or industrial applicability, may be produced according to the methods of the present invention. In some aspects, these proteins may be purified for use in pharmaceutical preparations. In other aspects, the transgenic cells themselves may be used therapeutically. For example, autologous cancer cells modified according to the methods of the present invention to express one or more immunostimulatory proteins may be reintroduced into the patient as a cancer vaccine. As another example, antigen presenting cells may be transfected according to the methods of the present invention to express one or more antigens and then introduced into a patient. The present invention could also be used for the production of viral vectors by transient co-transfection of cells.

1. Therapeutic Proteins

The transfected cells of the present invention are modified to express one or more therapeutic proteins. A “therapeutic protein” is a protein that can be administered to a subject for the purpose of treating or preventing a disease. Examples of classes of therapeutic proteins include tumor suppressors, inducers of apoptosis, cell cycle regulators, immuno-stimulatory proteins, toxins, cytokines, enzymes, antibodies, inhibitors of angiogenesis, metalloproteinase inhibitors, hormones or peptide hormones.

a. Immuno-Stimulatory Proteins

In some embodiments of the invention, the therapeutic protein is an immuno-stimulatory protein. An “immuno-stimulatory protein” is a protein involved in the activation, differentiation, or chemotaxis of immune cells. Examples of classes of immuno-stimulatory proteins include cytokines and thymic hormones. Thymic hormones include, for example, prothymosin-α, thymulin, thymic humoral factor (THF), THF-γ-2, thymocyte growth peptide (TGP), thymopoietin (TPO), thymopentin, and thymosin-α-1.

The term cytokine refers to a diverse group of secreted, soluble proteins and peptides that mediate communication among cells and modulate the functional activities of individual cells and tissues. Classes of cytokines include interleukins, interferons, colony stimulating factors, and chemokines. Examples of cytokines include: IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, leukocyte inhibitory factor (LIF), IFN-α, IFN-γ, TNF, TNF-α, TGF-β, G-CSF, M-CSF, and GM-CSF.

Interleukins are involved in processes of cell activation, cell differentiation, proliferation, and cell-to-cell interactions. Those of skill in the art are familiar with interleukins including, but not limited to: IL-1, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-17B, IL-17C, IL-17E, IL-17F, IL-18, IL-19, IL-20, IL-21, IL-21, IL-22, IL-23, IL-24, IL-25, IL-26, IL-27, IL-28A, IL-28B, IL-29, and IL-30.

Interferons are proteins that possess antiviral, antiproliferative, and immunomodulating activities. In addition, interferons influence metabolism, growth, and differentiation of cells. IFN-α, IFN-β, and IFN-γ are the three main human interferons. IFN-γ, which is produced primarily by the Th1 type of lymphocytes, exhibits many immunoregulatory effects, including the ability to induce the differentiation and activation of T cells and macrophages. Colony stimulating factors include, for example, G-CSF, M-CSF, GM-CSF, IL-3, and MEG-CSA.

Chemokines are a family of pro-inflammatory activation-inducible cytokines, which are mainly chemotactic for different cell types. There are four major classes of chemokines: C-chemokines, CC-chemokines, CXC-chemokines, and CX3C-chemokines. Non-limiting examples of chemokines include MCP-1, MCP-2, MCP-3, MIP-1α/β, IP-10, MIG, IL-8, RANTES, and lymphotactin. Other immuno-stimulatory proteins that may be used in the methods and compositions of the present invention include B7.1 (CD80), B7.2 (CD86), CD40, CD40 Ligand (CD40L), LFA-1, ICAM-1, VLA-4, and VCAM-1.

b. Developmental Proteins

Developmental proteins is another class of proteins whose expression may be enhanced by using the compositions and methods of the present invention. Developmental genes include, for example, adhesion molecules, cyclin kinase inhibitors, Wnt family members, Pax family members, Winged helix family members, Hox family members, cytokines/lymphokines and their receptors, growth or differentiation factors and their receptors, neurotransmitters and their receptors.

c. Oncogenes and Tumor Suppressors

The methods of the present invention can be used to produce oncogenes and tumor suppressors. Non-limiting examples of oncogenes include ABL1, BLC1, BCL6, CBFA1, CBL, CSFIR, ERBA, ERBB, EBRB2, ETS1, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, and YES. Non-limiting examples of tumor suppressor genes include p53, Rb, Rap1A, DCC, k-rev, BRCA1, BRCA2, zac1, p73, MMAC-1, ATM, HIC-1, DPC-4, FHIT, APC, DCC, PTEN, ING1, NOEY1, NOEY2, PML, OVCA1, MADR2, WT1, 53BP2, IRF-1, MADH4, MCC, NF1, NF2, RB1, TP53, and WT1.

d. Enzymes

Particularly appropriate genes for expression include enzyme-encoding genes. Enzymes are used for a wide-variety of therapeutic, research, commercial, and industrial purposes. Examples of useful gene products include carbamoyl synthetase I, ornithine transcarbamylase, arginosuccinate synthetase, arginosuccinate lyase, and arginase. Other desirable gene products include fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione β-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, β-glucosidase, pyruvate carboxylase, hepatic phosphorylase, phosphorylase kinase, glycine decarboxylase (also referred to as P-protein), H-protein, T-protein, Menkes disease copper-transporting ATPase, and Wilson's disease copper-transporting ATPase.

Other examples include cytosine deaminase, hypoxanthine-guanine phosphoribosyltransferase, galactose-1-phosphate uridylyltransferase, galactokinase, UDP-galactose-4-epimerase, phenylalanine hydroxylase, glucocerbrosidase, sphingomyelinase, α-L-iduronidase, glucose-6-phosphate dehydrogenase, HSV thymidine kinase, and human thymidine kinase,

Other types of enzymes include ACP desaturases and hycroxylases, ADP-glucose pyrophorylases, ATPases, alcohol dehydrogenases, amylases, amyloglucosidases, catalases, cellulases, cyclooxygenases, decarboxylases, dextrinases, esterases, DNA and RNA polymerases, hyaluron synthases, galactosidases, glucanases, glucose oxidases, GTPases, helicases, hemicellulases, hyaluronidases, integrases, invertases, isomersases, kinases, lactases, lipases, lipoxygenases, lyases, lysozymes, pectinesterases, peroxidases, phosphatases, phospholipases, phophorylases, polygalacturonases, proteinases and peptideases, pullanases, recombinases, reverse transcriptases, topoisomerases, and xylanases.

e. Hormones

Hormones are another group of genes that may be produced according to the methods described herein. Included are growth hormone, prolactin, placental lactogen, luteinizing hormone, follicle-stimulating hormone, chorionic gonadotropin, thyroid-stimulating hormone, leptin, adrenocorticotropin (ACTH), angiotensin I and II, β-endorphin, β-melanocyte stimulating hormone (β-MSH), cholecystokinin, endothelin I, galanin, gastric inhibitory peptide (GIP), glucagon, insulin, lipotropins, neurophysins, somatostatin, calcitonin, calcitonin gene related peptide (CGRP), β-calcitonin gene related peptide, hypercalcemia of malignancy factor, parathyroid hormone-related protein (PTH-rP), glucagon-like peptide (GLP-1), pancreastatin, pancreatic peptide, peptide YY, PHM, secretin, vasoactive intestinal peptide (VIP), oxytocin, vasopressin (AVP), vasotocin, enkephalinamide, metorphinamide, alpha melanocyte stimulating hormone (alpha-MSH), atrial natriuretic factor (ANF), amylin, amyloid P component (SAP-1), corticotropin releasing hormone (CRH), growth hormone releasing factor (GHRH), luteinizing hormone-releasing hormone (LHRH), neuropeptide Y, substance K (neurokinin A), substance P, and thyrotropin releasing hormone (TRH).

f. Antigens

A therapeutic protein can also be an antigenic peptide or polypeptide capable of generating an immune response. Examples include polynucleotides encoding antigens such as viral antigens, bacterial antigens, fungal antigens or parasitic antigens. Virus targets include picornavirus, coronavirus, togavirus, flavivirus, rhabdovirus, paramyxovirus, orthomyxovirus, bunyavirus, arenvirus, reovirus, retrovirus, papovavirus, parvovirus, herpesvirus, poxvirus, hepadnavirus, and spongiform virus. Parasite targets include trypanosomes, tapeworms, roundworms, and helminthes. Also, tumor markers, such as fetal antigen or prostate specific antigen, may be targeted in this manner.

g. Other Proteins

Other examples of proteins that can be produced according to the methods of the present invention include blood derivatives; growth factors; neurotransmitters or their precursors or synthetic enzymes; trophic factors (such as BDNF, CNTF, NGF, IGF, GMF, aFGF, bFGF, NT3, NT5, and the like); apolipoproteins (such as ApoAI, ApoAIV, ApoE, and the like); dystrophin or a minidystrophin; genes coding for factors involved in coagulation (such as factors VII, VIII, IX, and the like); cytosine deaminase, or all or part of a natural or artificial immunoglobulin (Fab, ScFv, and the like); anti-thrombotic genes (e.g., COX-1, TFPI); genes involved in angiogenesis (e.g., VEGF, FGF, thrombospondin, BAI-1, GDAIF, or their receptors); MCC, and mouse or humanized monoclonal antibodies.

2. Protein Purification

It may be desirable to purify the proteins produced according to the present invention. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography, polyacrylamide gel electrophoresis, and isoelectric focusing. A particularly efficient method of purifying peptides is fast protein liquid chromatography or HPLC.

Certain aspects of the present invention concern the purification, and in particular embodiments, the substantial purification, of an encoded protein or peptide. The purified proteins can be used in pharmaceutical compositions or for research, commercial, or industrial applications. The term “purified protein or peptide” as used herein, is intended to refer to a composition, isolatable from other components, wherein the protein or peptide is purified to any degree from the components of the cell in which it was produced. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

3. Transgenic Cells for Cell Therapy

The present invention can also be used to produce transgenic cells having enhanced transgene expression for use in cell therapy. With cell based therapy, cells are genetically modified ex vivo, and then reintroduced into the subject. The methods disclosed herein can be used to enhance transgene expression from these cells.

There are a variety of cell therapy approaches known in the art. Therapies involving secreted proteins are amenable to treatment using this approach. Exemplary secreted proteins include cytokines, colony stimulating factors, nerve growth factors, and hormones.

The methods described herein could be used to enhance transgene expression in genetically modified tumor cells. The tumor cells could be modified to overexpress one or more immuno-stimulatory proteins. Once transfected with the immuno-stimulatory protein(s), the cells could be irradiated, or otherwise inactivated, and administered to a cancer patient, in order to stimulate an immune response against the tumor cells. Methods and compositions relating to cancer vaccines are disclosed in the U.S. Provisional Patent Application entitled “Genetically Modified Tumor Cells as Cancer Vaccines” by Liu et al., filed Dec. 10, 2004, incorporated herein by reference.

The methods described herein could also be used to enhance the expression of antigens from antigen presenting cells. For example, antigen presenting cells could be loaded with nucleic acid vectors encoding one or more antigens ex vivo according the methods of the present invention. The transfected cells could then be administered to a patient in order to stimulate an immune response.

4. Viral Vector Production

The methods of the present invention are useful in the production of viral vectors. Viruses are highly efficient at nucleic acid delivery to specific cell types, while often avoiding detection by the infected host's immune system. These features make certain viruses attractive candidates as gene-delivery vehicles for use in gene therapies (Robbins and Ghivizzani, 1998; Cristiano et al., 1998).

Current transient transfection methods, such as CaPO₄ and small static electroporation, allow production of small volumes of viral vectors at a time, and the process is cumbersome, expensive and inefficient. Methods employing CaPO₄ can introduce inconsistencies from lot to lot, which can potentially lead to regulatory issues. Furthermore, precipitation of CaPO₄ interferes downstream purification/concentration of viral particles, which withholds some human clinical trials because a 1000-fold virus concentration is typically needed.

Scaling up of production for replication-incompetent viral vectors is a major hurdle for large gene therapy clinical trials. Transient, simultaneous transfection of cells with multiple plasmids results in the production of vectors and decreases the possibility of viral-genome recombination. The present invention provides a method of enhancing the expression of these plasmids, and thus provides a safe and reliable method, which can be used for large-scale viral vector production, including retrovirus, lentivirus, adenovirus, AAV, and alphavirus vector productions. The production of infectious vectors by electroporation-mediated co-transfection of cells is described in U.S. patent application Ser. No. 10/751,586, incorporated herein by reference.

M. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### AZA Effect on Transgene Expression of Cytosolic Protein in K562 Cells

5′-aza-2′-deoxycytidine (AZA) enhanced and prolonged the expression of the cytosolic protein, eGFP, under the control of either a CMV promoter or a pGK promoter following the electroporation-mediated transfection of K562 cells.

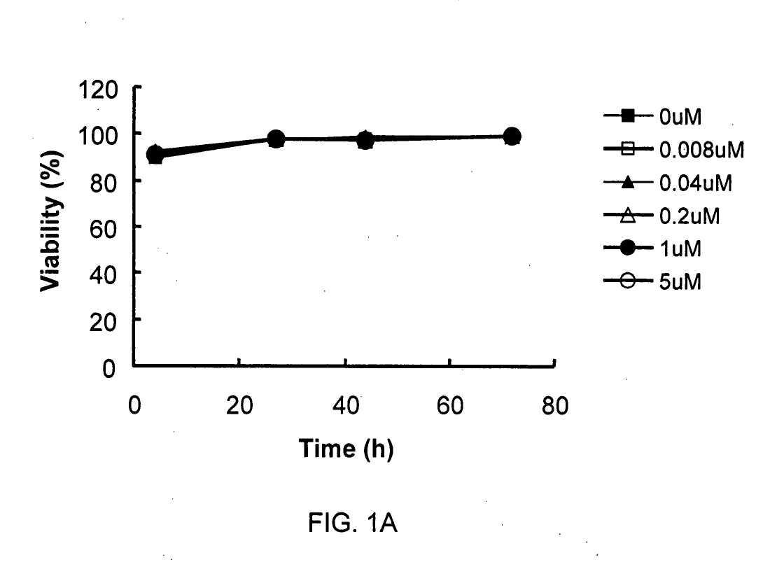

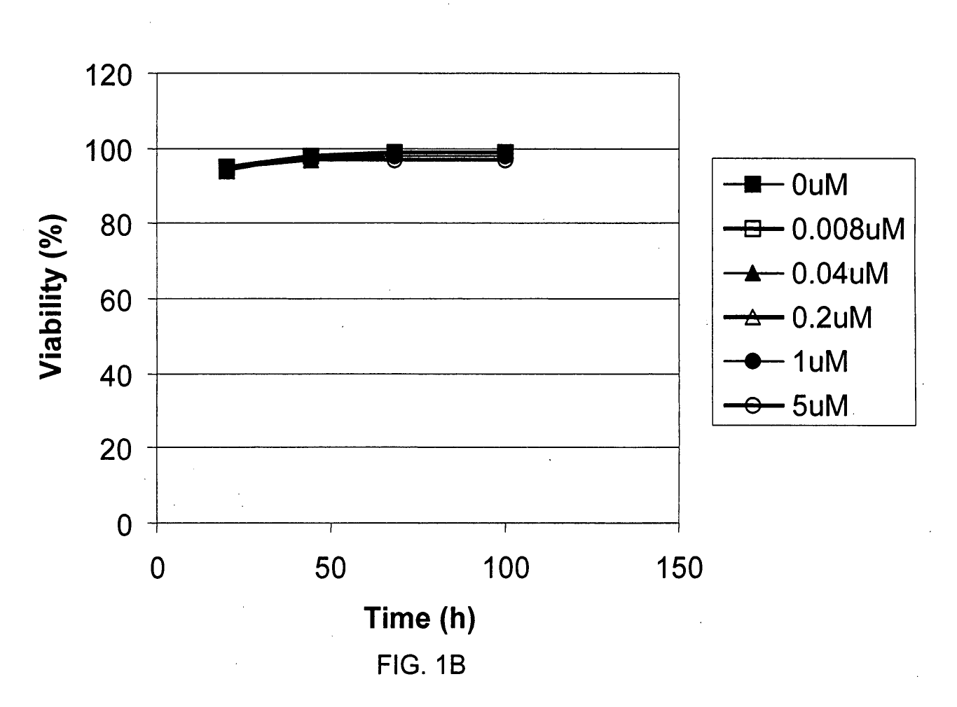

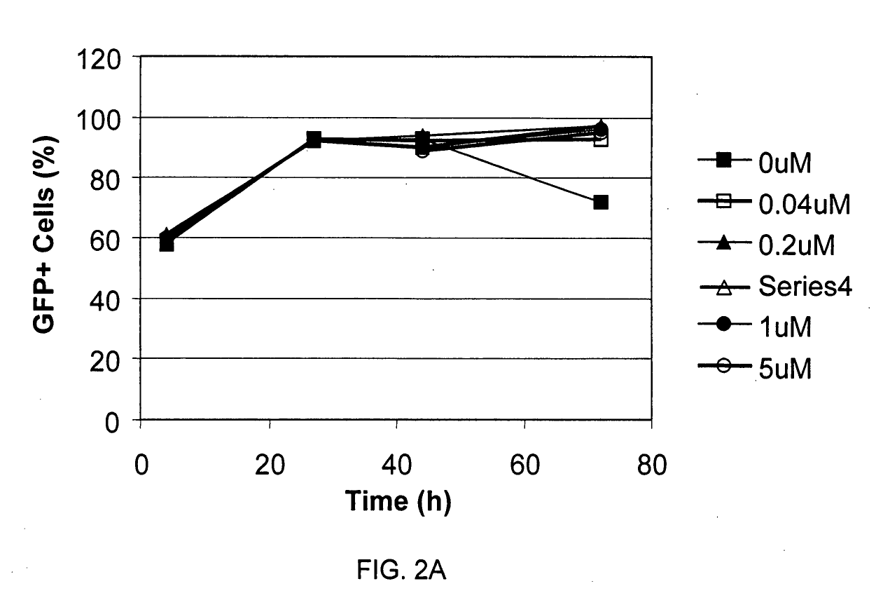

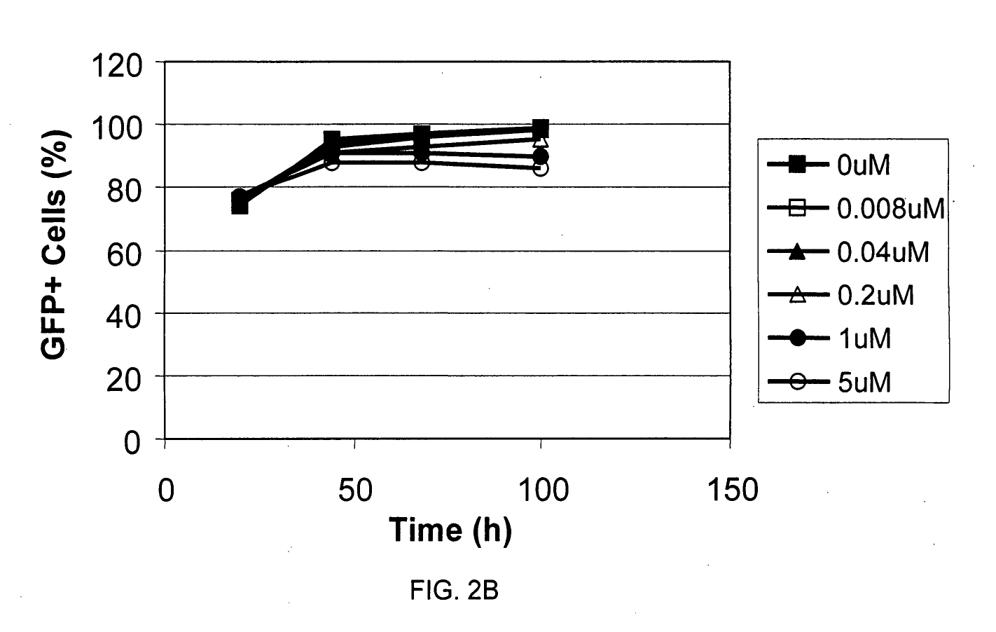

K562 cells were transfected by electroporation (1 kV/cm, four 400 μs pulses) with 200 μg/ml of the plasmid CMV-eGFP or PGK-eGFP. Cells were cultured post-transfection in full medium with 0.0, 0.008, 0.04, 0.2, 1.0, or 5.0 μM AZA. The culture medium and AZA was changed every 24 hours. Cells were analyzed by flow cytometry. Cell viability was assayed by PI exclusion test. As shown in FIGS. 1A and 1B, AZA had no effect on cell viability. Furthermore, at this expression level the percentage of GFP+ cells was not significantly dependent on AZA concentration (FIGS. 2A and 2B).

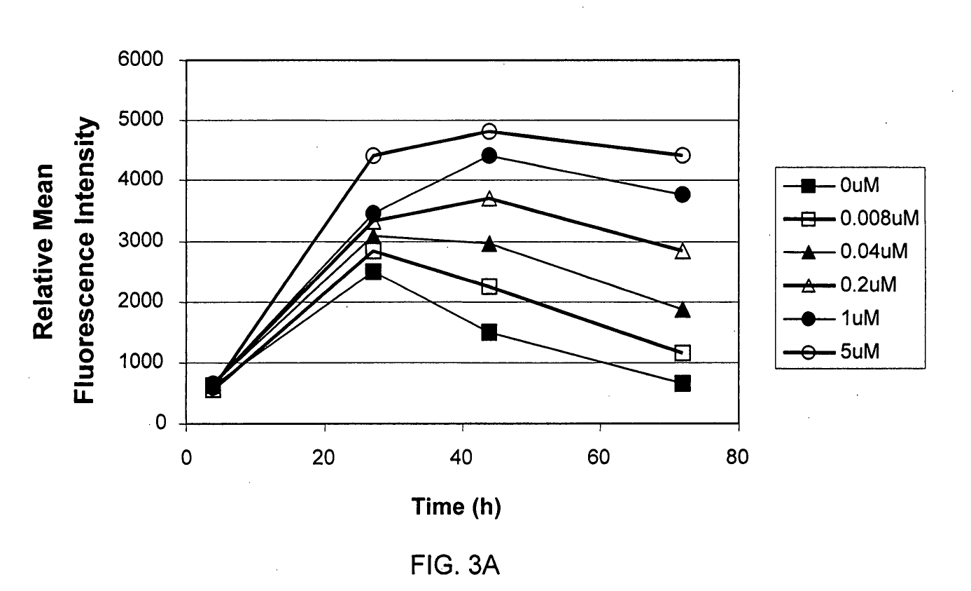

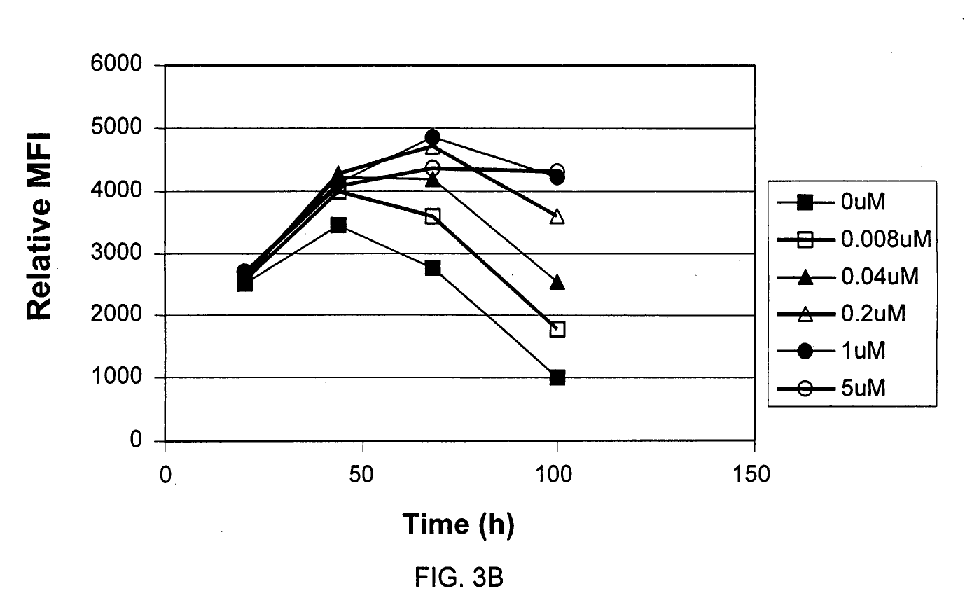

AZA did, however, have a dose-dependent effect on the rate of reduction of the decrease of the expression level of the transfected cells. As shown in FIGS. 3A and 3B, the highest tested concentrations of AZA maintained the highest levels of GFP expression. In cells transfected with the CMV-eGFP plasmid, the AZA effect was not significant with 5 hours of treatment, but became significant with longer treatment times. At about 24 hours the AZA effect is easily detected (FIG. 3A). The most significant AZA effect on mean fluorescent intensity in cells tranfected with CMV-eGFP in this study was at 72 hours, with a relative value of about 500 for untreated cells and a relative value of about 4,000 for cells treated with 5 μM AZA (FIG. 3A). In cells transfected with the PGK-eGFP plasmid, the AZA effect was not significant at 24 hours post-transfection, but became significant beginning at about 48 hours post-transfection (FIG. 3B). The most significant AZA effect on mean fluorescence intensity in cell transfected with PGK-eGFP for this study was at 100 hours post-transfection, with a relative value of about 1,000 for untreated cells and a relative value of about 4,000 for cells treated with 5 μM AZA (FIG. 3B).

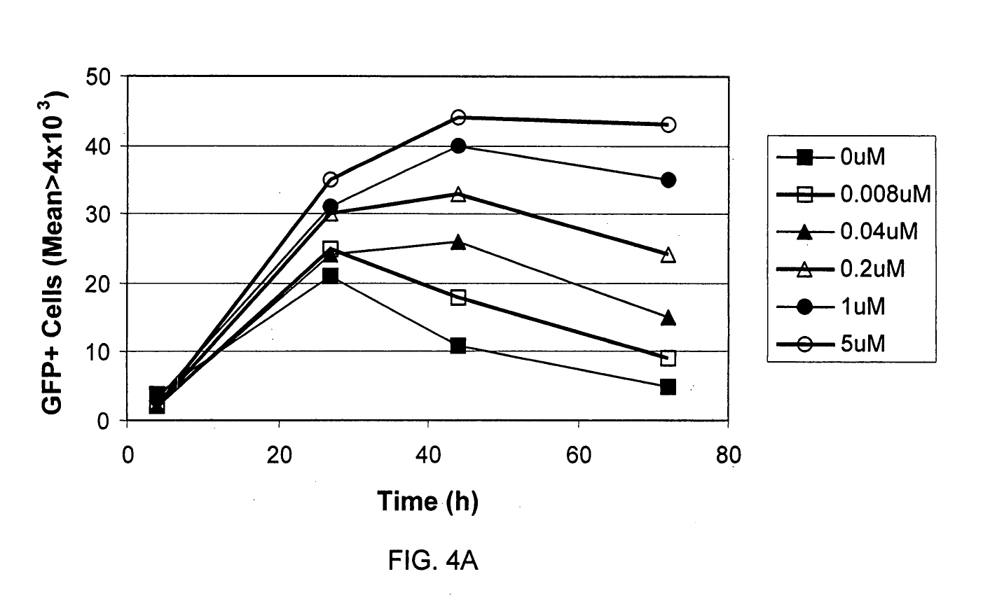

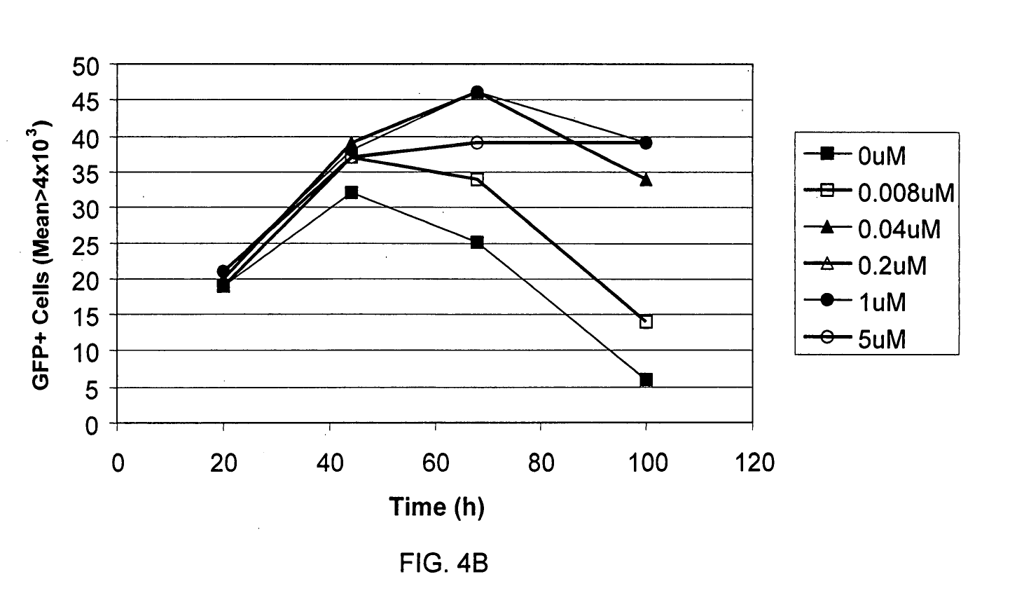

As shown in FIGS. 4A and 4B, AZA treatment also had a dose-dependent effect on the rate of reduction of the decrease of the percentage of the high-level GFP expressing cells (mean>4×10³). The higher the AZA concentration, the higher the percentage of high-level GFP expressing cells maintained in culture over time. In cells transfected with CMV-eGFP, the AZA effect was not significant with 5 hours treatment, but became significant at about 24 hours post-transfection (FIG. 4A). The most significant AZA effect on percentage of high-level expressing cells transfected with CMV-eGFP in this study was at 72 hours, with about 5% for untreated cells and 43% for cells treated with 5 μM AZA (FIG. 4A). In cells transfected with PGK-eGFP, the AZA effect became significant at about 24 hours post-transfection (FIG. 4B). The most significant AZA effect on percentage of high-level expressing cells transfected with PGK-eGFP in this study was at 100 hours, with about 5% for untreated cells and 40% for cells treated with 5 μM AZA (FIG. 4A).

### EXAMPLE 2

### AZA Effect on Transgene Expression of Membrane Protein in K562 Cells

AZA enhanced and prolonged the expression of the membrane protein, hCD40L, under the control of CMV promoter following the electroporation-mediated transfection of K562 cells.

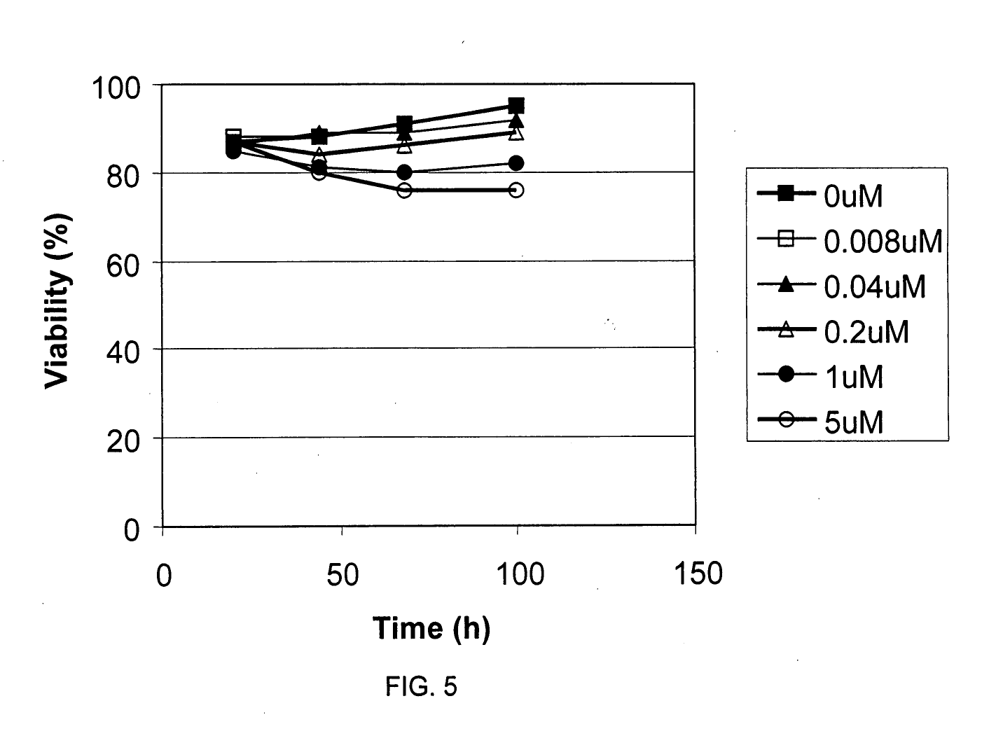

K562 cells were transfected by electroporation (1 kV/cm, four 400 μs pulses) with 200 μg/ml of the plasmid CMV-hCD40L. Cells were cultured post-transfection in full medium with 0.0, 0.008, 0.04, 0.2, 1.0, or 5.0 μM AZA. The culture medium and AZA was changed every 24 hours. Cells were analyzed by flow cytometry. Cell viability was assayed by PI exclusion test. As shown in FIG. 5, AZA had no significant effect on cell viability.

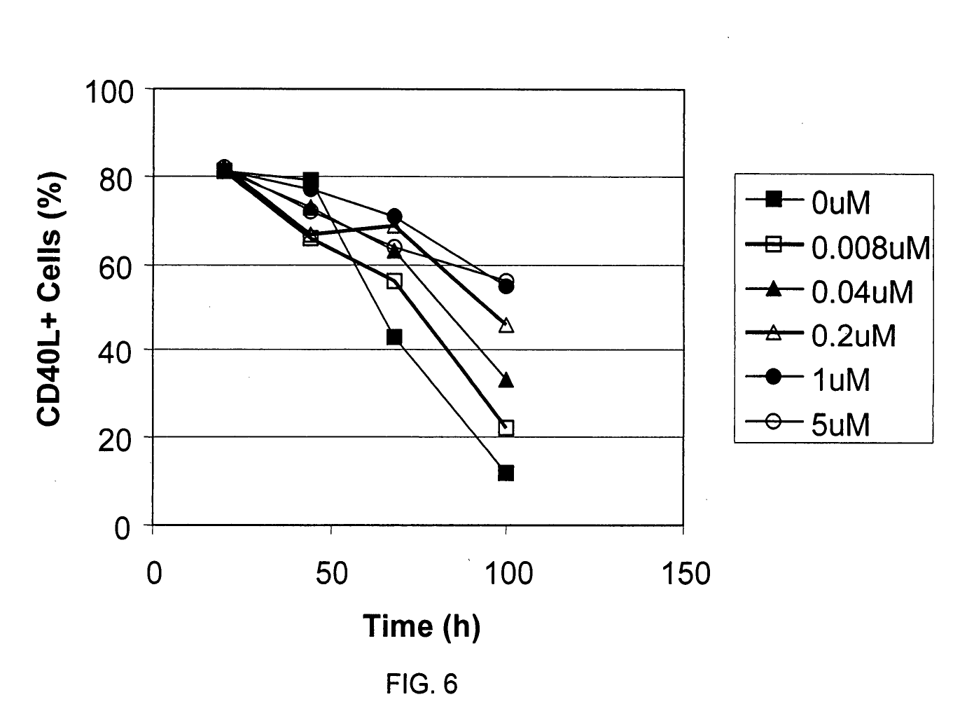

As shown in FIG. 6, AZA had a dose-dependent effect on slowing down the decrease of the hCD40L expression level of the transfected cells. The higher the AZA concentration, the higher the percentage of the hCD40L-expressing cells were maintained. The AZA effect became significant at about 48 hours post-transfection. The most significant AZA effect on the percentage of hCD40L-expressing cells for this study was at 100 hours post-transfection, with about 10% for untreated cells and 60% for cells treated with 5 μM AZA.

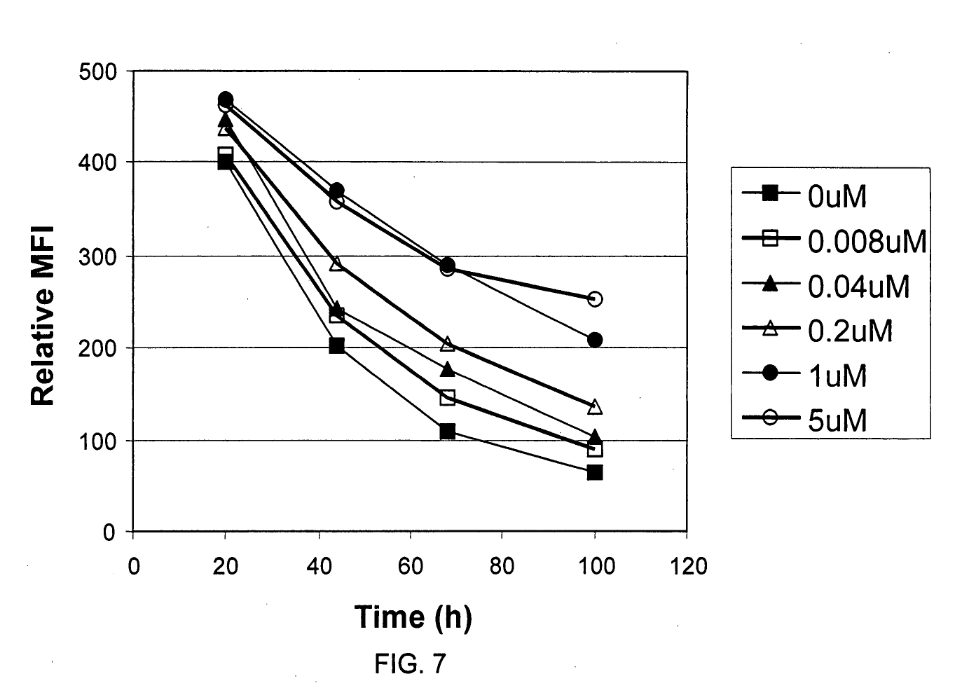

As shown in FIG. 7, AZA treatment also had a dose-dependent effect on slowing down the decrease of the expression level of transfected cells. The higher the AZA concentration, the higher the hCD40L expression levels the cells maintained. The AZA effect became significant at about 24 hours post-transfection. The most significant AZA effect on mean fluorescence intensity for this study was at 100 hours post-transfection, with relative units of about 80 for untreated cells and about 280 for cells treated with 5 μM AZA.

### EXAMPLE 3

### AZA Effect on Transgene Expression of Secreted Protein in K562 Cells

AZA enhanced and prolonged the expression of the secreted proteins, hIL-2 and mIL-12, under the control of CMV promoter following the electroporation-mediated transfection of K562 cells.

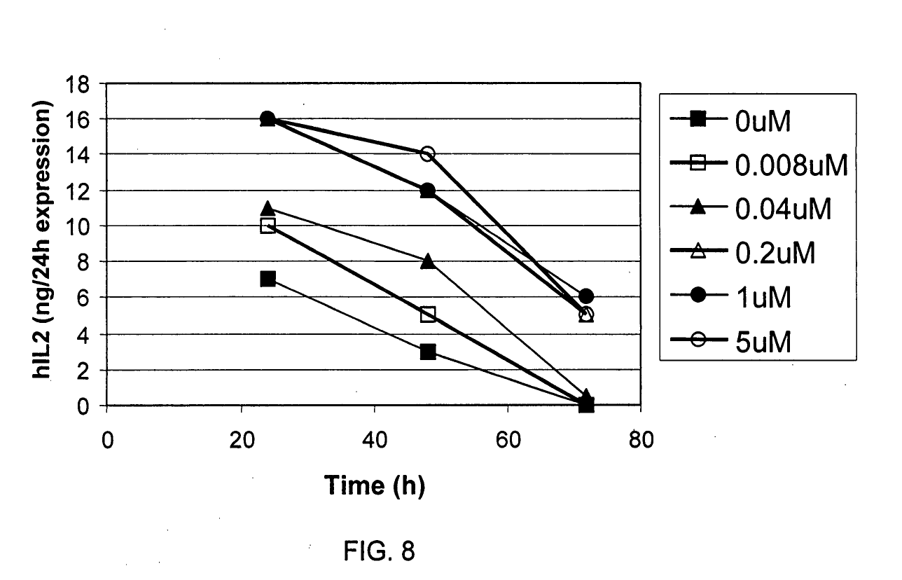

K562 cells were transfected by electroporation (1 kV/cm, four 400 us pulses) with 200 μg/ml of the plasmid CMV-hIL-2. Cells (1e5) were cultured post-transfection in full medium with 0.0, 0.008, 0.04, 0.2, 1.0, or 5.0 μM AZA. The secretion level of hIL-2 was analyzed by ELISA and presented as ng/24 h. As shown in FIG. 8, AZA had a dose-dependent effect on the rate of reduction of the decrease of the expression level of transfected cells. The higher the AZA concentration, the higher the hIL-2 expression level the cells maintained. The AZA effect became significant at about 24 hours post-transfection. The most significant AZA effect on hIL-2 secretion for this study was at 72 hours post-transfection, with about 0 ng/24 h for untreated cells and about 5 ng/24 h for cells treated with 5 μM AZA.

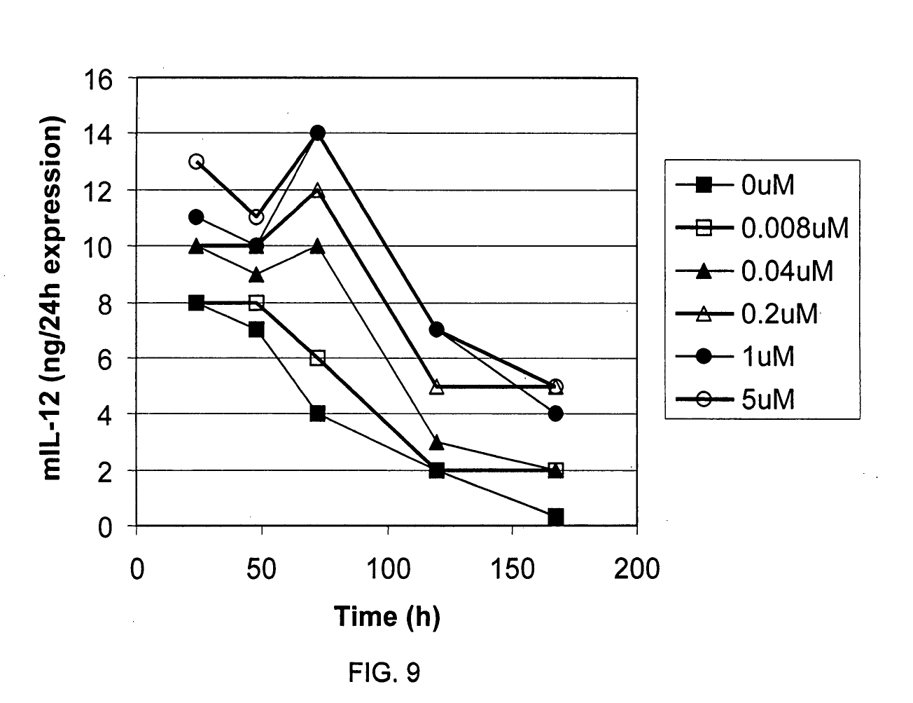

K562 cells were transfected by electroporation (1 kV/cm, four 400 μs pulses) with 200 μg/ml of the plasmid CMV-mIL-12. Cells (1e5) were cultured post-transfection in full medium with 0.0, 0.008, 0.04, 0.2, 1.0, or 5.0 μM AZA. The secretion level of mIL-12 was analyzed by ELISA and presented as ng/24 h. As shown in FIG. 9, AZA had a dose-dependent effect on the rate of reduction of the decrease of the expression level of transfected cells. The higher the AZA concentration, the higher the mIL-12 expression level the cells maintained. The AZA effect became significant beginning at about 24 hours post-transfection. The most significant AZA effect on hIL-2 secretion for this study was at 168 hours post-transfection, with about 0 ng/24 h for untreated cells and about 5 ng/24 h for cells treated with 5 μM AZA.

### EXAMPLE 4

### AZA Effect on Transgene Expression of Cytosolic Protein in 293T Cells

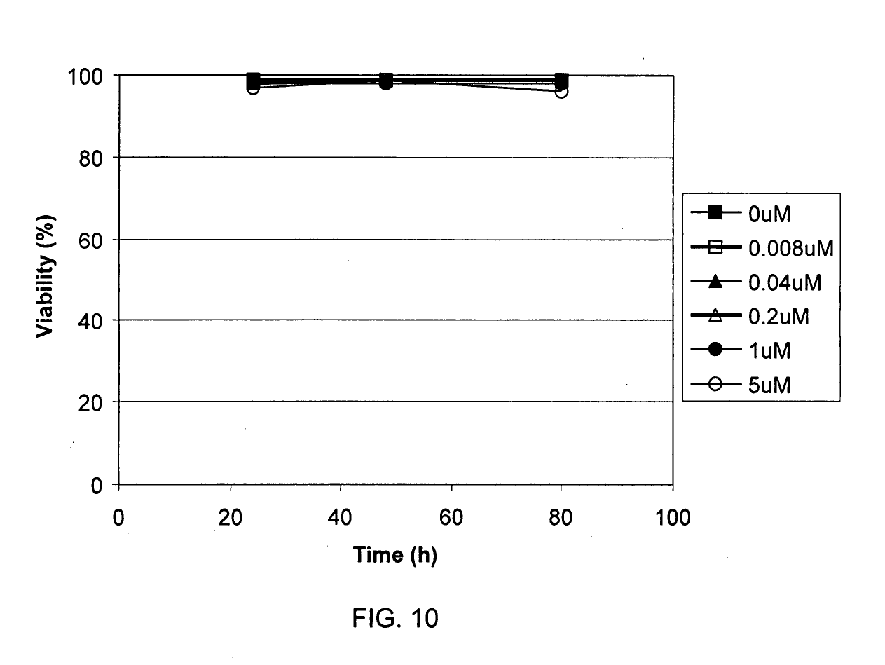

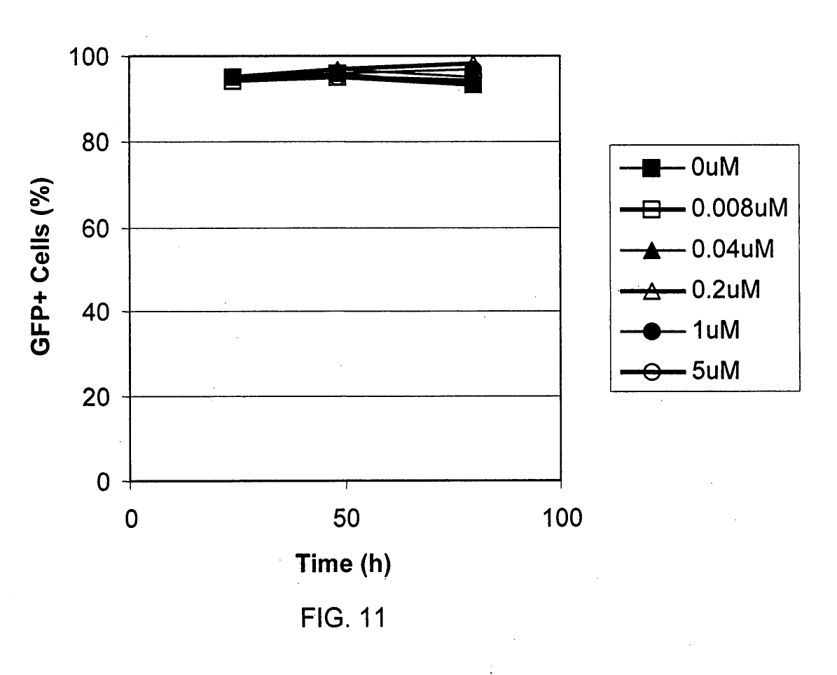

293T cells were transfected by electroporation (1 kV/cm, four 400 μs pulses) with 200 μg/ml of the plasmid CMV-eGFP. Cells were cultured post-transfection in full medium with 0.0, 0.008, 0.04, 0.2, 1.0, or 5.0 μM AZA. The culture medium and AZA was changed every 24 hours. Cells were analyzed by flow cytometry. Cell viability was assayed by PI exclusion test. As shown in FIG. 10, AZA had no effect on cell viability. Furthermore, the percentage of GFP+ cells was not significantly dependent on AZA concentration at this expression level (FIG. 11).

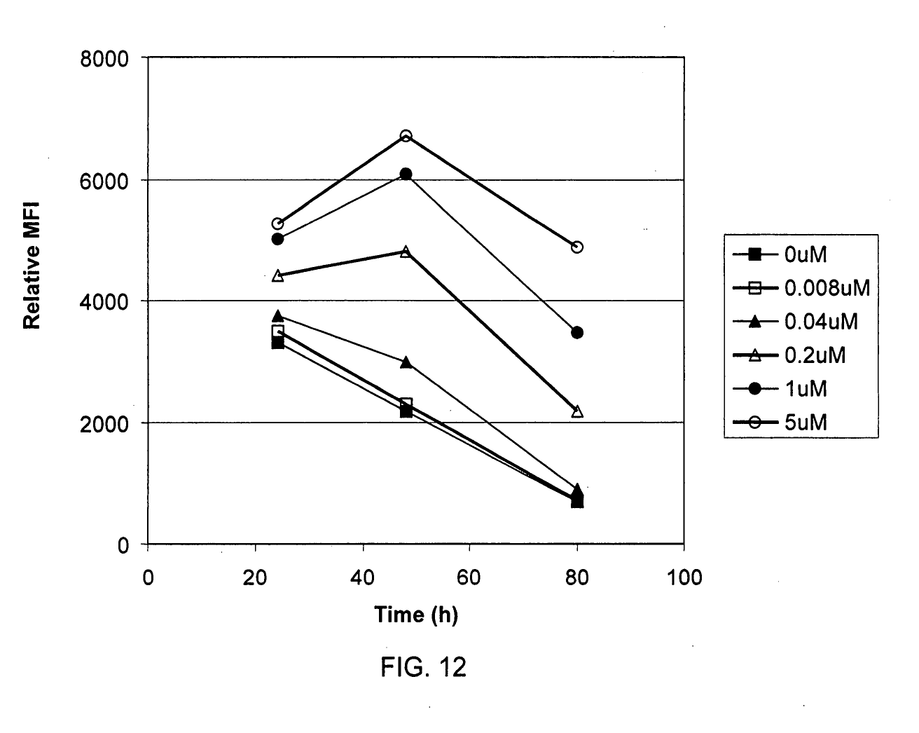

As shown in FIG. 12, AZA had a dose-dependent effect on slowing down the decrease of the expression level of transfected 293T cells. The higher the AZA concentration, the higher the eGFP expression level the cells maintained. The AZA effect became significant at about 24 hours post-transfection. The most significant AZA effect on mean fluorescence intensity for this study was at 80 hours post-transfection, with a relative value of about 500 for untreated cells and a relative value of about 5,000 for cells treated with 5 μM AZA.

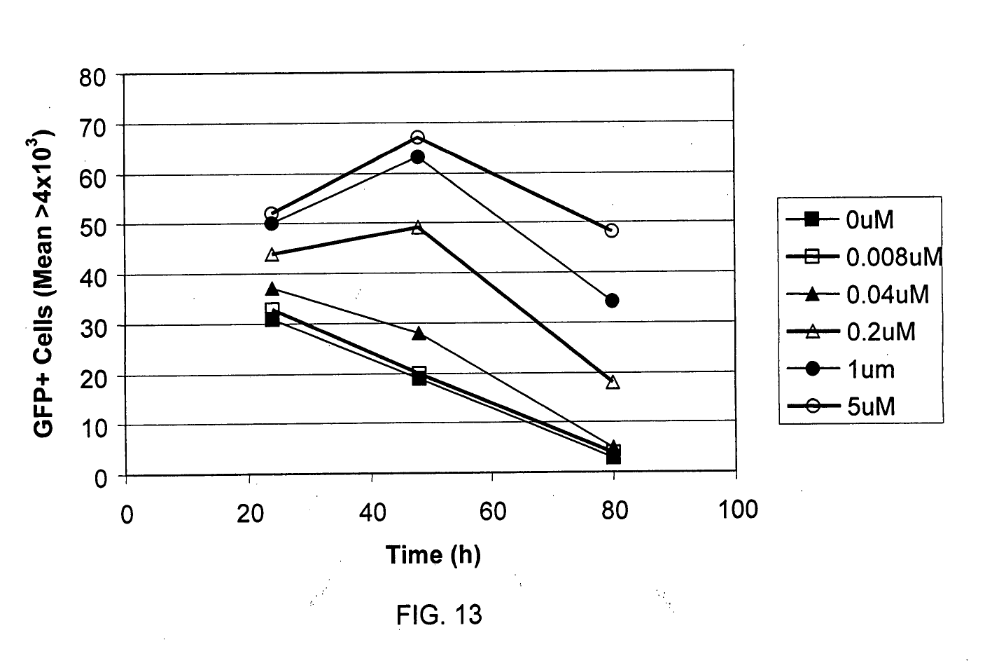

As shown in FIG. 13, AZA had a dose-dependent effect on slowing down the decrease of the percentage of the high-level GFP expressing cells (mean>4×10³). The higher the AZA concentration, the higher the percentage of high-level GFP-expressing cells maintained. The AZA effect became significant at about 24 hours post-transfection. The most significant AZA effect on percentage of high-level eGFP expressing cells for this study was at 80 hours post-transfection, with about 5% for untreated cells and about 50% for cells treated with 5 μM AZA.

### EXAMPLE 5

### AZA Effect on Transgene Expression of Secreted Protein in 293T Cells

AZA enhanced and prolonged the expression of the secreted protein, mIL-12, under the control of CMV promoter following the electroporation-mediated transfection of 293T cells.

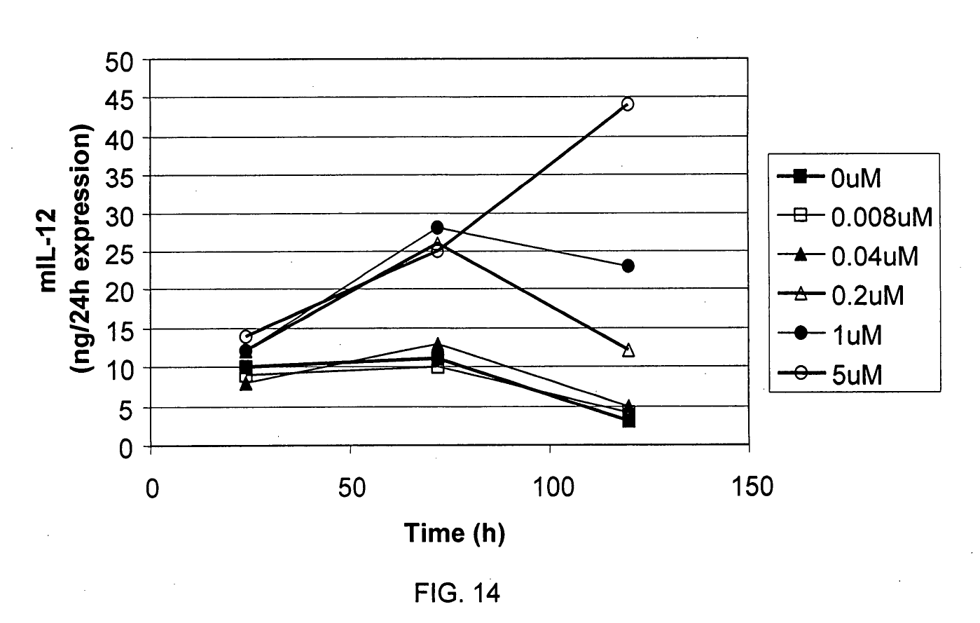

293T cells were transfected by electroporation (1 kV/cm, four 400 μs pulses) with 200 μg/ml of the plasmid CMV-mIL-12. Cells (3e4) were cultured post-transfection in full medium with 0.0, 0.008, 0.04, 0.2, 1.0, or 5.0 μM AZA. The secretion level of mIL-12 was analyzed by ELISA and presented as ng/24 h. As shown in FIG. 14, AZA had a dose-dependent effect on the rate of reduction of the decrease of the expression level of transfected cells. The higher the AZA concentration, the higher the mIL-12 expression level the cells maintained. The AZA effect became significant at about 24 hours post-transfection. The most significant AZA effect on mIL-12 secretion for this study was at 120 hours post-transfection, with about 4 ng/24 h for untreated cells and about 45 ng/24 h for cells treated with 5 μM AZA.

### EXAMPLE 6

AZA Effect on Cell Proliferation of Transfected K562 Cells

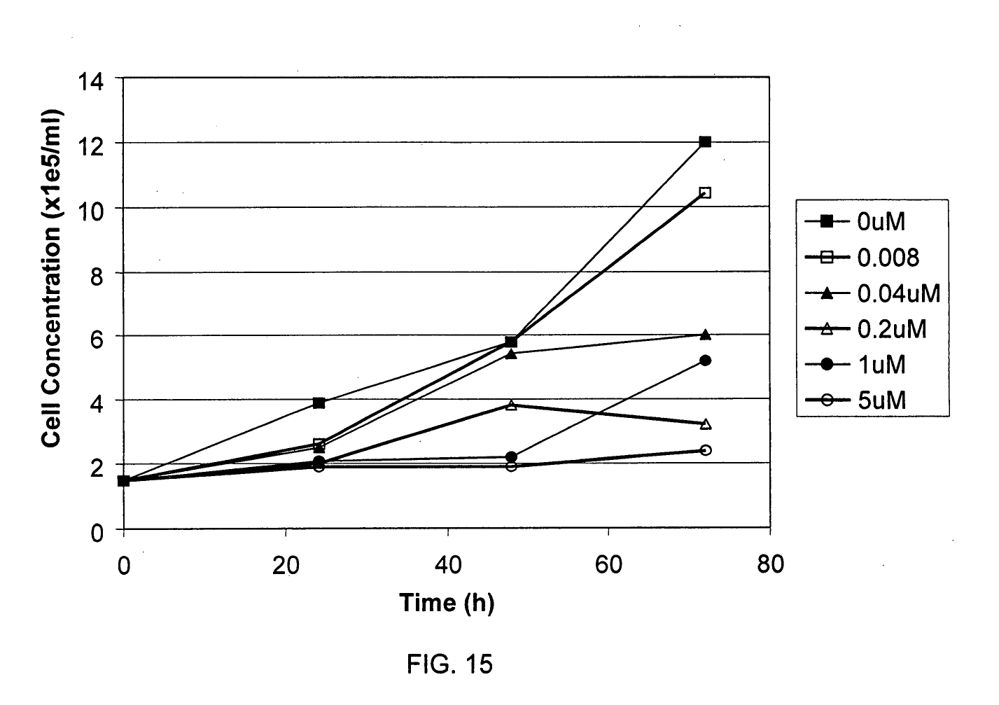

K562 cells were transfected by electroporation (1 kV/cm, four 400 μs pulses) with 200 μg/ml of the plasmid CMV-eGFP. Cells were cultured post-transfection in full medium with 0.0, 0.008, 0.04, 0.2, 1.0, or 5.0 μM AZA. The cell concentration was analyzed by hemacytometer. As shown in FIG. 15, AZA retarded cell growth in a dose-dependent manner. The cells did not grow significantly when treated with 5 μM of AZA.

### EXAMPLE 7

### AZA Effect on the Morphology of Transfected Cells

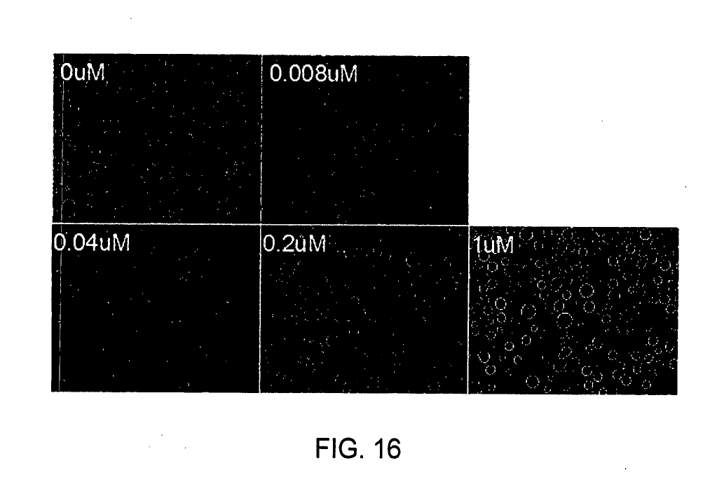

K562 cells were transfected by electroporation (1 kV/cm, four 400 μs pulses) with 200 μg/ml of the plasmid CMV-eGFP. Cells were cultured for 7 days post-transfection in full medium with the AZA concentrations indicated in FIG. 16. The morphology of the cells was recorded. AZA treatment caused an increase in cell size, cell death, and a decrease of cell proliferation.

### EXAMPLE 8

### Pre-Transfection AZA Treatment of Transfected Cells

K562 cells were cultured overnight in complete medium with μM AZA prior to transfection. Cells were transfected by electroporation (1 kV/cm, four 400 μs pulses) with 200 μg/ml of the plasmid CMV-eGFP. Each transfected cell sample was divided into two parts and cultured in medium either without additional AZA or with 5 μM AZA. Cells were analyzed by flow cytometry at 4.5, 22 and 48 hours post transfection.

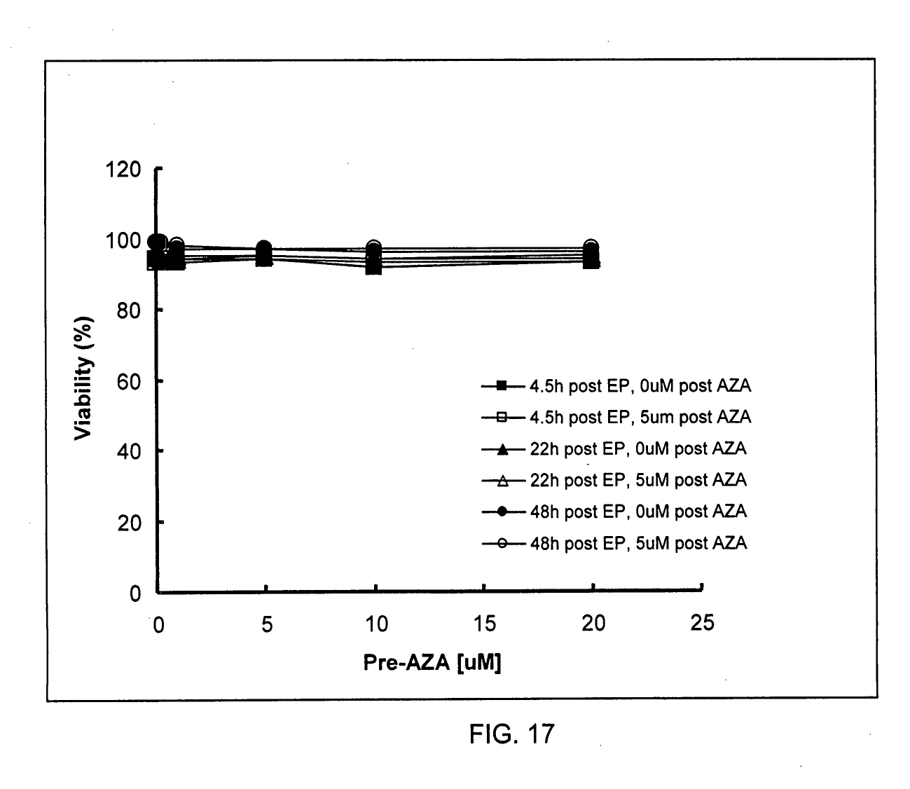

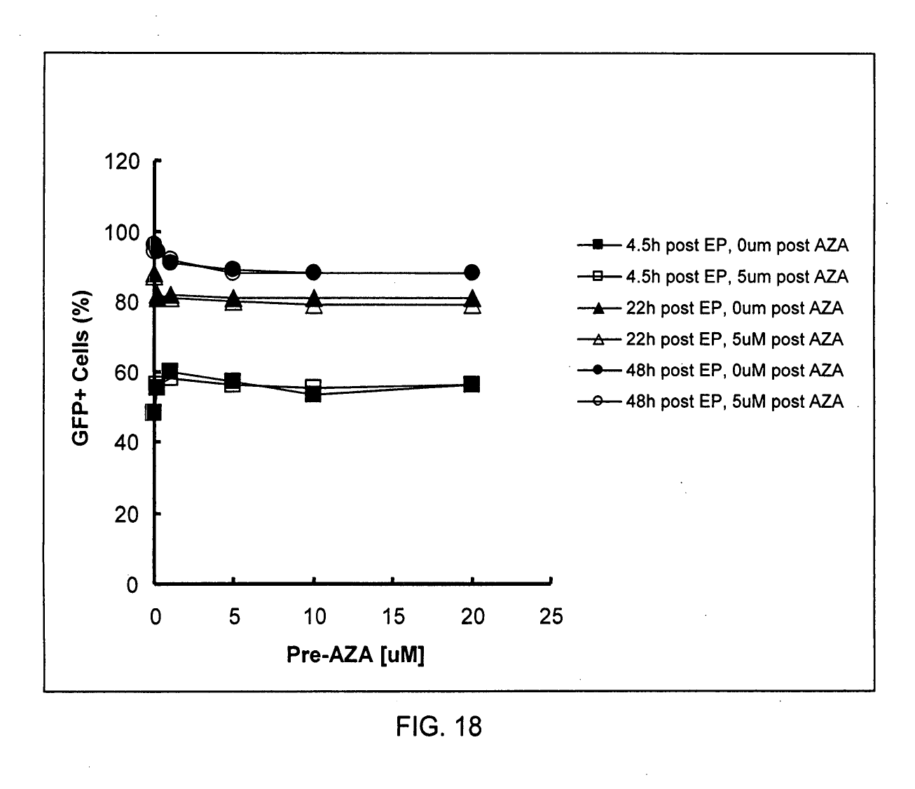

Cell viability was assayed by PI exclusion using flow cytometry. As shown in FIG. 17, treatment with AZA had no effect on cell viability. Transfection efficiency (GFP+ cells) was assayed by flow cytometric analysis. As shown in FIG. 18, even though the percentage of GFP+ cells increases with time after transfection, pre- and post-transfection AZA treatment had no significant effect on the percentage of GFP+ cells at this expression level.

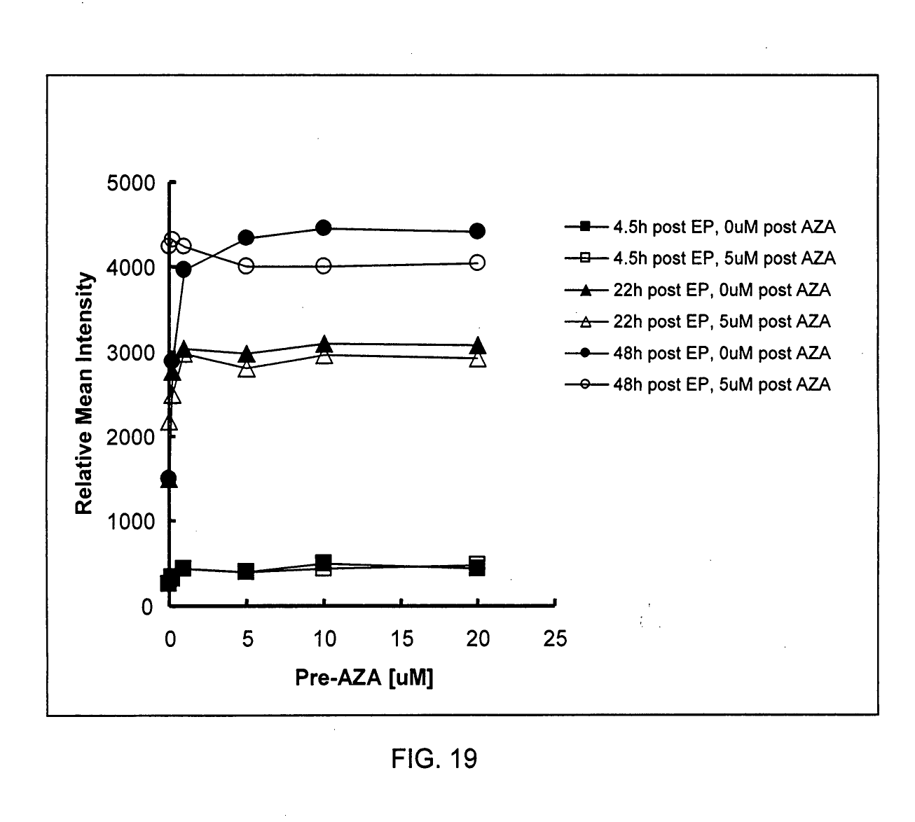

As shown in FIG. 19, mean fluorescence intensity (MFI) increases with AZA concentration in the pre-transfection culture medium from 0 μM to 5 μM AZA, and plateaus beyond 5 μM AZA when there is no AZA in the post-transfection culture medium. When 5 μM AZA was added to the culture medium following transfection, the MFI became independent of the pre-transfection AZA concentration, except at the 4.5 hour post-transfection time point (see FIG. 19). When the pre-transfection AZA concentration was 5 μM or above, the post-transfection AZA treatment did not further increase significantly the MFI.

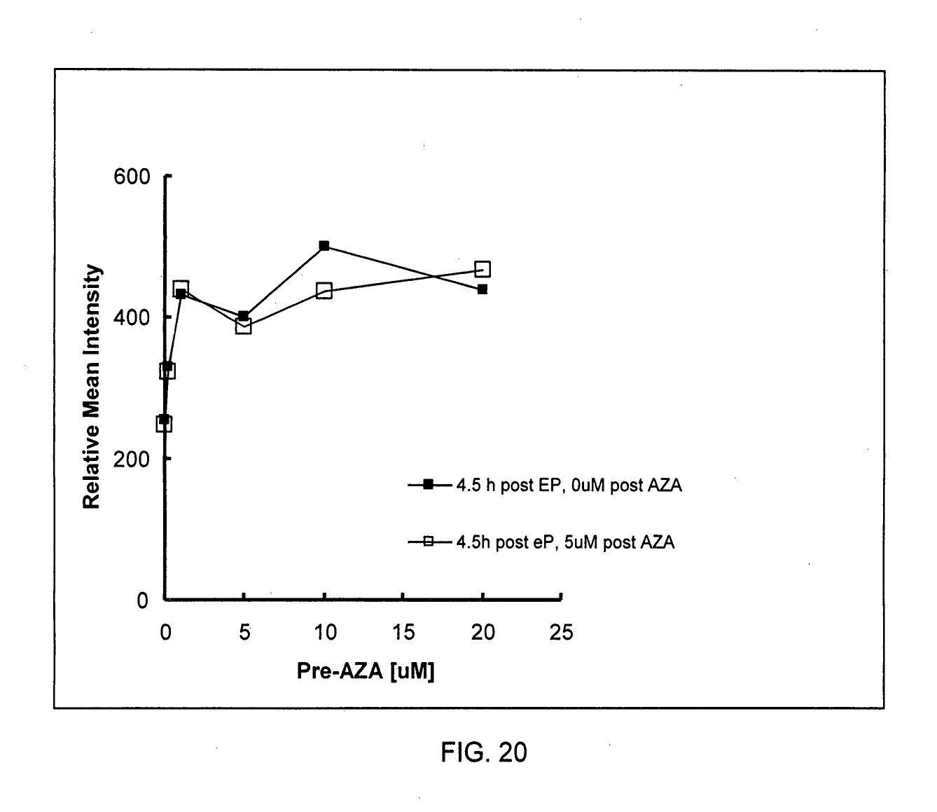

FIG. 20 shows a more detailed view of the 4.5 hour time point samples shown in FIG. 19. Mean fluorescence intensity (MFI) increases with pre-transfection AZA dose up to 5 μM AZA either with or without post-transfection AZA treatment (FIG. 20). The MFI with 5 μM and above pre-transfection AZA is about twice that with no pre-transfection AZA either with or without 5 μM post-transfection AZA treatment.

### EXAMPLE 9

### AZA Improves Viral Vector Production

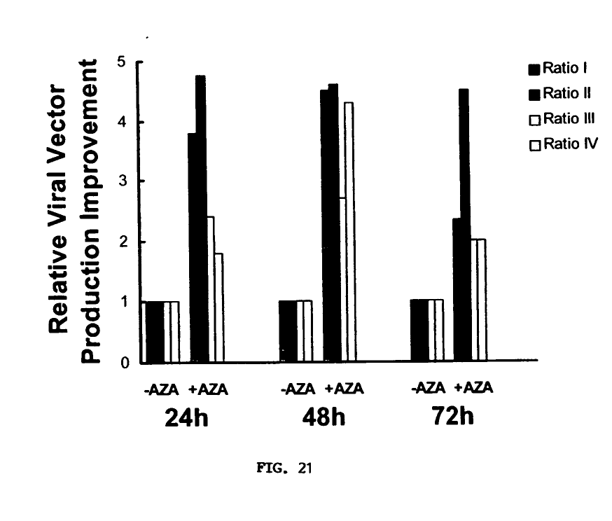

Three plasmids coding for a transgene (eGFP), lentiviral viral gag/pol, and the viral envelop protein VSVG were co-transfected by electroporation into 293T cells with DNA ratios I, II, III and IV (eGFP: gag/pol: VSVG ratios of 50:20:0.625, 50:20:1.25, 50:20:2.5 and 50:20:5, respectively) to produce viral particles. Each transfected sample was divided into two parts following transfection and grown in culture medium with 5 μM AZA (+AZA) or without AZA (−AZA). Virus-containing supernatant was collected at 24 hours, 48 hours, and 72 hours post transfection and were titered using D17 cells. As shown in FIG. 21, viral vector production was improved for all DNA ratios when the cells were cultured in the presence of AZA following transfection.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. application Ser. No. 10/781,440
U.S. application Ser. No. 10/751,586
U.S. application Ser. No. 10/675,592
U.S. application Ser. No. 10/225,446
U.S. application Ser. No. 10/080,272
U.S. Pat. No. 4,352,883
U.S. Pat. No. 4,683,202
U.S. Pat. No. 5,612,207
U.S. Pat. No. 5,720,921
U.S. Pat. No. 5,928,906
U.S. Pat. No. 6,074,605
U.S. Pat. No. 6,090,617
U.S. Pat. No. 6,485,961
U.S. Pat. No. 6,617,154
U.S. Pat. No. 6,773,669
Almendro et al., J. Immunol., 157(12):5411-5421, 1996.
Angel et al., Mol Cell. Biol., 7:2256, 1987a.
Angel et al., Cell, 49:729, 1987b.
Atchison and Perry, Cell, 46:253, 1986.
Atchison and Perry, Cell, 48:121, 1987.
Ausubel et al., In: Current Protocols in Molecular Biology, John, Wiley & Sons, Inc, New York, 1996.
Banerji et al., Cell, 27(2 Pt 1):299-308, 1981.
Banerji et al., Cell, 33(3):729-740, 1983.
Berkhout et al., Cell, 59:273-282, 1989.
Blanar et al., EMBO J., 8:1139, 1989.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1983.
Carbonelli et al., FEMS Microbiol. Lett., 177(1):75-82, 1999.
Celander and Haseltine, J. Virology, 61:269, 1987.
Celander et al., J. Virology, 62:1314, 1988.
Chandler et al., Cell, 33:489, 1983.
Chandler et al., Proc. Natl. Acad. Sci. USA, 94(8):3596-601, 1997.
Chang et al., Mol. Cell. Biol., 9:2153, 1989.
Chatterjee et al., Proc. Natl. Acad. Sci. USA, 86:9114, 1989.
Choi et al., Cell, 53:519, 1988.
Cocea, Biotechniques, 23(5):814-816, 1997.
Cohen et al., J. Cell. Physiol., 5:75, 1987.
Costa et al., Mol. Cell. Biol., 8:81, 1988.
Cripe et al., EMBO J., 6:3745, 1987.
Cristiano et al., Cancer Detect. Prev., 22(5):445-454, 1998.
Culotta and Hamer, Mol. Cell. Biol., 9:1376, 1989.
D'Angelo et al., Mol. Endocrinol., 13(5):692-704, 1999.
Dandolo et al., J. Virology, 47:55-64, 1983.
De Villiers et al., Nature, 312(5991):242-246, 1984.
Deschamps et al., Science, 230:1174-1177, 1985.
Edbrooke et al., Mol. Cell. Biol., 9:1908, 1989.
Edlund et al., Science, 230:912-916, 1985.
Feng and Holland, Nature, 334:6178, 1988.
Firak and Subramanian, Mol. Cell. Biol., 6:3667, 1986.
Foecking and Hofstetter, Gene, 45(1):101-105, 1986.
Fujita et al., Cell, 49:357, 1987.
Gabriel and Teissie, Biophys. J., 76(4):2158-2165, 1999.
Gilles et al., Cell, 33:717, 1983.
Gloss et al., EMBO J., 6:3735, 1987.
Godbout et al., Mol. Cell. Biol., 8:1169, 1988.
Goodbourn and Maniatis, Proc. Natl. Acad. Sci. USA, 85:1447, 1988.
Goodbourn et al., Cell, 45:601, 1986.
Greene et al., Immunology Today, 10:272, 1989
Grosschedl and Baltimore, Cell, 41:885, 1985.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Herr and Clarke, Cell, 45:461, 1986.
Hibino et al., Biophys. J., 64(6):1789-1800, 1993.
Hirochika et al., J. Virol., 61:2599, 1987.
Hirsch et al., Mol. Cell. Biol., 10:1959, 1990.
Holbrook et al., Virology, 157:211, 1987.
Horlick and Benfield, Mol. Cell. Biol., 9:2396, 1989.
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9:3393, 1989.
Kadesch and Berg, Mol. Cell. Biol., 6:2593, 1986.
Kakorin et al., Biophys. Chem., 58(1-2):109-116, 1996.
Karin et al., Mol. Cell. Biol., 7:606, 1987.
Katinka et al., Cell, 20:393, 1980.
Kawamoto et al., Mol. Cell. Biol., 8:267, 1988.
Kiledjian et al., Mol. Cell. Biol., 8:145, 1988.
Klamut et al., Mol. Cell. Biol., 10: 193, 1990.
Koch et al., Mol. Cell. Biol., 9:303, 1989.
Kotnik et al., Bioenerg., 43:281-291; 45:3-16, 1998.
Kraus et al. FEBS Lett., 428(3):165-170, 1998.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
Lareyre et al., J. Biol. Chem., 274(12):8282-8290, 1999.
Larsen et al., Proc Natl. Acad. Sci. USA., 83:8283, 1986.
Laspia et al., Cell, 59:283, 1989.
Latimer et al., Mol. Cell. Biol., 10:760, 1990.
Lee et al., Biochem. Biophys. Res. Commun., 240(2):309-313, 1997.
Lee et al., Nature, 294:228, 1981.
Lee et al., Nucleic Acids Res., 12:4191-206, 1984.
Levenson et al., Hum. Gene Ther., 9(8):1233-1236, 1998.
Levinson et al., Nature, 295:79, 1982.
Lin et al., Mol. Cell. Biol., 10:850, 1990.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Samow, Nature, 353:90-94, 1991.
MacGregor et al., Am. J. Med., 82(3B): 16-22, 1987.
Majors and Varmus, Proc. Natl. Acad. Sci. USA, 80:5866, 1983.
McNeall et al., Gene, 76:81, 1989.
Miksicek et al., Cell, 46:203, 1986.
Mir, Bioelectrochem., 53:1-10, 2000.
Mizrahi, Dev. Biol. Stand., 55:219-230, 1983.
Mordacq and Linzer, Genes and Dev., 3:760, 1989.
Moreau et al., Nucl. Acids Res., 9:6047, 1981.
Muesing et al., Cell, 48:691, 1987.
Neumann et al., Proc. Natl. Acad. Sci. USA, 96(16):9345-9350, 1999.
Ng et al., Nuc. Acids Res., 17:601, 1989.
Nomoto et al., Gene, 236(2):259-271, 1999.
Ondek et al., EMBO J., 6:1017, 1987.
Ornitz et al., Mol. Cell. Biol., 7:3466, 1987.
Palmiter et al., Nature, 300:611, 1982.
PCT Appln. 94/17178
PCT Appln. WO 03/018751
PCT Appln. WO 04/031353
Pech et al., Mol. Cell. Biol., 9:396, 1989.
Pelletier and Sonenberg, Nature, 334(6180):320-325, 1988.
Perez-Stable and Constantini, Mol. Cell. Biol., 10:1116, 1990.
Phillips et al., In: Large Scale Mammalian Cell Culture, Feder and Tolbert (Eds.), Academic Press, Orlando, Fla., U.S.A., 1985.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10: 1076, 1990.
Queen and Baltimore, Cell, 35:741, 1983.
Quinn et al., Mol. Cell. Biol., 9:4713, 1989.
Redondo et al., Science, 247:1225, 1990.
Reisman and Rotter, Mol. Cell. Biol., 9:3571, 1989.
Resendez Jr., et al., Mol. Cell. Biol., 8:4579, 1988.
Ripe et al., Mol. Cell. Biol., 9:2224, 1989.
Rittling et al., Nuc. Acids Res., 17:1619, 1989.
Robbins and Ghivizzani, Pharmacol Ther, 80(1):35-47, 1998.
Rols and Teissie, Biophys. J., 75(3):1415-1423, 1998.
Rosen et al., Cell, 41:813, 1988.
Sakai et al., Genes and Dev., 2:1144, 1988.
Sambrook et al., In: Molecular cloning, Cold Spring Harbor Laboratory Press, Cold Spring
Harbor, N.Y., 2001.
Satake et al., J. Virology, 62:970, 1988.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Thiesen et al., J. Virology, 62:614, 1988.
Treisman, Cell, 42:889, 1985.
Tronche et al., Mol. Biol. Med., 7:173, 1990.
Trudel and Constantini, Genes and Dev., 6:954, 1987.
Tsumaki et al., J. Biol. Chem., 273(36):22861-22864, 1998.
Tyndell et al., Nuc. Acids. Res., 9:6231, 1981.
Van Wezel, Nature, 216(110):64-65, 1967.
Vannice and Levinson, J. Virology, 62:1305, 1988.
Vasseur et al., Proc Natl. Acad. Sci. USA, 77:1068, 1980.
Wang and Calame, Cell, 47:241, 1986.
Weaver and Chizmadzhev, Bioenerg., 41:135-160, 1996.
Weber et al., Cell, 36:983, 1984.
Weinberger et al. Mol. Cell. Biol., 8:988, 1984.
Winoto and Baltimore, Cell, 59:649, 1989.
Wu et al., Biochem. Biophys. Res. Commun., 233(1):221-226, 1997.
Yutzey et al. Mol. Cell. Biol., 9:1397, 1989.
Zhao-Emonet et al., Biochim. Biophys. Acta, 1442(2-3):109-119, 1998.

What is claimed is:

1. A method for enhancing transgene expression in a eukaryotic cell, the method comprising:
(a) transfecting a eukaryotic cell by electroporation with an expression construct encoding a transgene; and
(b) contacting the cell with a methyltransferase inhibitor,
wherein the expression of the transgene is enhanced as compared to the expression of the transgene in a second eukaryotic cell not contacted with a methyltransferase inhibitor.

2. The method of claim 1, wherein the electroporation is static electroporation.

3. The method of claim 1, wherein the electroporation is flow electroporation.

4. The method of claim 1, wherein the cell is contacted with the methyltransferase inhibitor prior to transfection with the expression construct.

5. The method of claim 1, wherein the cell is contacted with the methyltransferase inhibitor during transfection with the expression construct.

6. The method of claim 1, wherein the cell is contacted with the methyltransferase inhibitor following transfection with the expression construct.

7. The method of claim 1, wherein enhancing transgene expression is further defined as increasing the level of transgene expression.

8. The method of claim 1, wherein enhancing transgene expression is further defined as prolonging transgene expression.

9. The method of claim 1, wherein enhancing transgene expression is further defined as increasing the level of transgene expression and prolonging transgene expression.

10. The method of claim 1, wherein the methyltransferase inhibitor is selected from the group consisting of 5-azacytidine, 5-aza-2′-deoxycytidine, L-ethionine, and 2-pyrimidone-1-β-D-riboside-1-(β-D-ribofuranosyl)-1,2-dihydropyrimidin-2-one (Zebularine).

11. The method of claim 10, wherein the methyltransferase inhibitor is an siRNA that targets an RNA encoding a DNA methyltransferase.

12. The method of claim 11, wherein the siRNA or a molecule encoding the siRNA is co-transfected with the expression construct.

13. The method of claim 1, wherein the transgene encodes a protein.

14. The method of claim 13, wherein the protein is a therapeutic protein.

15. The method of claim 1, wherein the cell is a K562 cell or a 293T cell.

16. The method of claim 1, wherein the expression construct comprises a CMV promoter or a PGK promoter.

17. The method of claim 1, wherein the transgene encodes a non-protein coding RNA.
